SEQUENCE LISTING (25 sequences)

SEQ ID NO: 1 - DNA, 40 bp, Homo sapiens
aagatgaattacagaaaaagatccaagaacttcagaaaaa

SEQ ID NO: 2 - DNA, 40 bp, Homo sapiens
taagggaaatgatagctagagaccgacagaaccaccaagt

SEQ ID NO: 3 - DNA, 40 bp, Homo sapiens
aagatgaattacagaaaaaggaccgacagaaccaccaagt

SEQ ID NO: 4 - DNA, 40 bp, Homo sapiens
aaactaaaaaagatcaagagaatgaactcagttcaaaagt

SEQ ID NO: 5 - DNA, 40 bp, Homo sapiens
aaactaaaaaagatcaagaggaaagcattgaagacccaaa

SEQ ID NO: 6 - DNA, 7704 bp, Homo sapiens
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagatgcaactaattcatgaaatttcgaacttaaagaatttagttaagcatcgagaagtatataatcaagatcttgagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagttgaaggagaaaatgaaagaacttgcaactacatacaagcaaatggaaaatgatattcagttatatcaaagccaattggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctccgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaaaaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaacttaatataaaacaaattagtgaggttcaggaaaacgtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcaagaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaaggaccgacagaaccaccaagtaaaacctgaaaaaaggttactaagtgatggacaacagcaccttatggaaagcctgagagaaaagtgctctagaataaaagagcttttgaagagatactcagagatggatgatcattatgagtgcttgaatagattgtctcttgacttggagaaggaaattgaattccacagaatcatgaagaaactgaagtatgtgttaagctatgttacaaaaataaaagaagaacaacatgaatgcatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcag

SEQ ID NO: 7 - protein, 2568 aa, Homo sapiens
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTDEALLKRYRKEIMDLKKQLEEVSLETRAQAMEKDQLAQLLEEKDLLQKVQNEKIENLTRMLVTSSSLTLQQELKAKRKRRVTWCLGKINKMKNSNYADQFNIPTNITTKTHKLSINLLREIDESVCSESDVFSNTLDTLSEIEWNPATKLLNQENIESELNSLRADYDNLVLDYEQLRTEKEEMELKLKEKNDLDEFEALERKTKKDQEMQLIHEISNLKNLVKHREVYNQDLENELSSKVELLREKEDQIKKLQEYIDSQKLENIKMDLSYSLESIEDPKQMKQTLFDAETVALDAKRESAFLRSENLELKEKMKELATTYKQMENDIQLYQSQLEAKKKMQVDLEKELQSAFNEITKLTSLIDGKVPKDLLCNLELEGKITDLQKELNKEVEENEALREEVILLSELKSLPSEVERLRKEIQDKSEELHIITSEKDKLFSEVVHKESRVQGLLEEIGKTKDDLATTQSNYKSTDQEFQNFKTLHMDFEQKYKMVLEENERMNQEIVNLSKEAQKFDSSLGALKTELSYKTQELQEKTREVQERLNEMEQLKEQLENRDSPLQTVEREKTLITEKLQQTLEEVKTLTQEKDDLKQLQESLQIERDQLKSDIHDTVNMNIDTQEQLRNALESLKQHQETINTLKSKISEEVSRNLHMEENTGETKDEFQQKMVGIDKKQDLEAKNTQTLTADVKDNEIIEQQRKIFSLIQEKNELQQMLESVIAEKEQLKTDLKENIEMTIENQEELRLLGDELKKQQEIVAQEKNHAIKKEGELSRTCDRLAEVEEKLKEKSQQLQEKQQQLLNVQEEMSEMQKKINEIENLKNELKNKELTLEHMETERLELAQKLNENYEEVKSITKERKVLKELQKSFETERDHLRGYIREIEATGLQTKEELKIAHIHLKEHQETIDELRRSVSEKTAQIINTQDLEKSHTKLQEEIPVLHEEQELLPNVKKVSETQETMNELELLTEQSTTKDSTTLARIEMERLRLNEKFQESQEEIKSLTKERDNLKTIKEALEVKHDQLKEHIRETLAKIQESQSKQEQSLNMKEKDNETTKIVSEMEQFKPKDSALLRIEIEMLGLSKRLQESHDEMKSVAKEKDDLQRLQEVLQSESDQLKENIKEIVAKHLETEEELKVAHCCLKEQEETINELRVNLSEKETEISTIQKQLEAINDKLQNKIQEIYEKEEQLNIKQISEVQENVNELKQFKEHRKAKDSALQSIESKMLELTNRLQESQEEIQIMIKEKEEMKRVQEALQIERDQLKENTKEIVAKMKESQEKEYQFLKMTAVNETQEKMCEIEHLKEQFETQKLNLENIETENIRLTQILHENLEEMRSVTKERDDLRSVEETLKVERDQLKENLRETITRDLEKQEELKIVHMHLKEHQETIDKLRGIVSEKTNEISNMQKDLEHSNDALKAQDLKIQEELRIAHMHLKEQQETIDKLRGIVSEKTDKLSNMQKDLENSNAKLQEKIQELKANEHQLITLKKDVNETQKKVSEMEQLKKQIKDQSLTLSKLEIENLNLAQELHENLEEMKSVMKERDNLRRVEETLKLERDQLKESLQETKARDLEIQQELKTARMLSKEHKETVDKLREKISEKTIQISDIQKDLDKSKDELQKKDRQNHQVKPEKRLLSDGQQHLMESLREKCSRIKELLKRYSEMDDHYECLNRLSLDLEKEIEFHRIMKKLKYVLSYVTKIKEEQHECINKFEMDFIDEVEKQKELLIKIQHLQQDCDVPSRELRDLKLNQNMDLHIEEILKDFSESEFPSIKTEFQQVLSNRKEMTQFLEEWLNTRFDIEKLKNGIQKENDRICQVNNFFNNRIIAIMNESTEFEERSATISKEWEQDLKSLKEKNEKLFKNYQTLKTSLASGAQVNPTTQDNKNPHVTSRATQLTTEKIRELENSLHEAKESAMHKESKIIKMQKELEVTNDIIAKLQAKVHESNKCLEKTKETIQVLQDKVALGAKPYKEEIEDLKMKLVKIDLEKMKNAKEFEKEISATKATVEYQKEVIRLLRENLRRSQQAQDTSVISEHTDPQPSNKPLTCGGGSGIVQNTKALILKSEHIRLEKEISKLKQQNEQLIKQKNELLSNNQHLSNEVKTWKERTLKREAHKQVTCENSPKSPKVTGTASKKKQITPSQCKERNLQDPVPKESPKSCFFDSRSKSLPSPHPVRYFDNSSLGLCPEVQNAGAESVDSQPGPWHASSGKDVPECKTQ

SEQ ID NO: 8 - DNA, 7629 bp, Homo sapiens
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagttgaaggagaaaatgaaagaacttgcaactacatacaagcaaatggaaaatgatattcagttatatcaaagccaattggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctccgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaaaaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaacttaatataaaacaaattagtgaggttcaggaaaacgtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcaagaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaaggaccgacagaaccaccaagtaaaacctgaaaaaaggttactaagtgatggacaacagcaccttatggaaagcctgagagaaaagtgctctagaataaaagagcttttgaagagatactcagagatggatgatcattatgagtgcttgaatagattgtctcttgacttggagaaggaaattgaattccacagaatcatgaagaaactgaagtatgtgttaagctatgttacaaaaataaaagaagaacaacatgaatgcatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcag

SEQ ID NO: 9 - protein, 2543 aa, Homo sapiens
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTDEALLKRYRKEIMDLKKQLEEVSLETRAQAMEKDQLAQLLEEKDLLQKVQNEKIENLTRMLVTSSSLTLQQELKAKRKRRVTWCLGKINKMKNSNYADQFNIPTNITTKTHKLSINLLREIDESVCSESDVFSNTLDTLSEIEWNPATKLLNQENIESELNSLRADYDNLVLDYEQLRTEKEEMELKLKEKNDLDEFEALERKTKKDQENELSSKVELLREKEDQIKKLQEYIDSQKLENIKMDLSYSLESIEDPKQMKQTLFDAETVALDAKRESAFLRSENLELKEKMKELATTYKQMENDIQLYQSQLEAKKKMQVDLEKELQSAFNEITKLTSLIDGKVPKDLLCNLELEGKITDLQKELNKEVEENEALREEVILLSELKSLPSEVERLRKEIQDKSEELHIITSEKDKLFSEVVHKESRVQGLLEEIGKTKDDLATTQSNYKSTDQEFQNFKTLHMDFEQKYKMVLEENERMNQEIVNLSKEAQKFDSSLGALKTELSYKTQELQEKTREVQERLNEMEQLKEQLENRDSPLQTVEREKTLITEKLQQTLEEVKTLTQEKDDLKQLQESLQIERDQLKSDIHDTVNMNIDTQEQLRNALESLKQHQETINTLKSKISEEVSRNLHMEENTGETKDEFQQKMVGIDKKQDLEAKNTQTLTADVKDNEIIEQQRKIFSLIQEKNELQQMLESVIAEKEQLKTDLKENIEMTIENQEELRLLGDELKKQQEIVAQEKNHAIKKEGELSRTCDRLAEVEEKLKEKSQQLQEKQQQLLNVQEEMSEMQKKINEIENLKNELKNKELTLEHMETERLELAQKLNENYEEVKSITKERKVLKELQKSFETERDHLRGYIREIEATGLQTKEELKIAHIHLKEHQETIDELRRSVSEKTAQIINTQDLEKSHTKLQEEIPVLHEEQELLPNVKKVSETQETMNELELLTEQSTTKDSTTLARIEMERLRLNEKFQESQEEIKSLTKERDNLKTIKEALEVKHDQLKEHIRETLAKIQESQSKQEQSLNMKEKDNETTKIVSEMEQFKPKDSALLRIEIEMLGLSKRLQESHDEMKSVAKEKDDLQRLQEVLQSESDQLKENIKEIVAKHLETEEELKVAHCCLKEQEETINELRVNLSEKETEISTIQKQLEAINDKLQNKIQEIYEKEEQLNIKQISEVQENVNELKQFKEHRKAKDSALQSIESKMLELTNRLQESQEEIQIMIKEKEEMKRVQEALQIERDQLKENTKEIVAKMKESQEKEYQFLKMTAVNETQEKMCEIEHLKEQFETQKLNLENIETENIRLTQILHENLEEMRSVTKERDDLRSVEETLKVERDQLKENLRETITRDLEKQEELKIVHMHLKEHQETIDKLRGIVSEKTNEISNMQKDLEHSNDALKAQDLKIQEELRIAHMHLKEQQETIDKLRGIVSEKTDKLSNMQKDLENSNAKLQEKIQELKANEHQLITLKKDVNETQKKVSEMEQLKKQIKDQSLTLSKLEIENLNLAQELHENLEEMKSVMKERDNLRRVEETLKLERDQLKESLQETKARDLEIQQELKTARMLSKEHKETVDKLREKISEKTIQISDIQKDLDKSKDELQKKDRQNHQVKPEKRLLSDGQQHLMESLREKCSRIKELLKRYSEMDDHYECLNRLSLDLEKEIEFHRIMKKLKYVLSYVTKIKEEQHECINKFEMDFIDEVEKQKELLIKIQHLQQDCDVPSRELRDLKLNQNMDLHIEEILKDFSESEFPSIKTEFQQVLSNRKEMTQFLEEWLNTRFDIEKLKNGIQKENDRICQVNNFFNNRIIAIMNESTEFEERSATISKEWEQDLKSLKEKNEKLFKNYQTLKTSLASGAQVNPTTQDNKNPHVTSRATQLTTEKIRELENSLHEAKESAMHKESKIIKMQKELEVTNDIIAKLQAKVHESNKCLEKTKETIQVLQDKVALGAKPYKEEIEDLKMKLVKIDLEKMKNAKEFEKEISATKATVEYQKEVIRLLRENLRRSQQAQDTSVISEHTDPQPSNKPLTCGGGSGIVQNTKALILKSEHIRLEKEISKLKQQNEQLIKQKNELLSNNQHLSNEVKTWKERTLKREAHKQVTCENSPKSPKVTGTASKKKQITPSQCKERNLQDPVPKESPKSCFFDSRSKSLPSPHPVRYFDNSSLGLCPEVQNAGAESVDSQPGPWHASSGKDVPECKTQ

SEQ ID NO: 10 - DNA, 7509 bp, Homo sapiens
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagaggaaagcattgaagacccaaaacaaatgaagcagactctgtttgatgctgaaactgtagcccttgatgccaagagagaatcagcctttcttagaagtgaaaatctggagttgaaggagaaaatgaaagaacttgcaactacatacaagcaaatggaaaatgatattcagttatatcaaagccaattggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctccgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaaaaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaacttaatataaaacaaattagtgaggttcaggaaaacgtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcaagaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaaggaccgacagaaccaccaagtaaaacctgaaaaaaggttactaagtgatggacaacagcaccttatggaaagcctgagagaaaagtgctctagaataaaagagcttttgaagagatactcagagatggatgatcattatgagtgcttgaatagattgtctcttgacttggagaaggaaattgaattccacagaatcatgaagaaactgaagtatgtgttaagctatgttacaaaaataaaagaagaacaacatgaatgcatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcag

SEQ ID NO: 11 - protein, 2503 aa, Homo sapiens
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTDEALLKRYRKEIMDLKKQLEEVSLETRAQAMEKDQLAQLLEEKDLLQKVQNEKIENLTRMLVTSSSLTLQQELKAKRKRRVTWCLGKINKMKNSNYADQFNIPTNITTKTHKLSINLLREIDESVCSESDVFSNTLDTLSEIEWNPATKLLNQENIESELNSLRADYDNLVLDYEQLRTEKEEMELKLKEKNDLDEFEALERKTKKDQEESIEDPKQMKQTLFDAETVALDAKRESAFLRSENLELKEKMKELATTYKQMENDIQLYQSQLEAKKKMQVDLEKELQSAFNEITKLTSLIDGKVPKDLLCNLELEGKITDLQKELNKEVEENEALREEVILLSELKSLPSEVERLRKEIQDKSEELHIITSEKDKLFSEVVHKESRVQGLLEEIGKTKDDLATTQSNYKSTDQEFQNFKTLHMDFEQKYKMVLEENERMNQEIVNLSKEAQKFDSSLGALKTELSYKTQELQEKTREVQERLNEMEQLKEQLENRDSPLQTVEREKTLITEKLQQTLEEVKTLTQEKDDLKQLQESLQIERDQLKSDIHDTVNMNIDTQEQLRNALESLKQHQETINTLKSKISEEVSRNLHMEENTGETKDEFQQKMVGIDKKQDLEAKNTQTLTADVKDNEIIEQQRKIFSLIQEKNELQQMLESVIAEKEQLKTDLKENIEMTIENQEELRLLGDELKKQQEIVAQEKNHAIKKEGELSRTCDRLAEVEEKLKEKSQQLQEKQQQLLNVQEEMSEMQKKINEIENLKNELKNKELTLEHMETERLELAQKLNENYEEVKSITKERKVLKELQKSFETERDHLRGYIREIEATGLQTKEELKIAHIHLKEHQETIDELRRSVSEKTAQIINTQDLEKSHTKLQEEIPVLHEEQELLPNVKKVSETQETMNELELLTEQSTTKDSTTLARIEMERLRLNEKFQESQEEIKSLTKERDNLKTIKEALEVKHDQLKEHIRETLAKIQESQSKQEQSLNMKEKDNETTKIVSEMEQFKPKDSALLRIEIEMLGLSKRLQESHDEMKSVAKEKDDLQRLQEVLQSESDQLKENIKEIVAKHLETEEELKVAHCCLKEQEETINELRVNLSEKETEISTIQKQLEAINDKLQNKIQEIYEKEEQLNIKQISEVQENVNELKQFKEHRKAKDSALQSIESKMLELTNRLQESQEEIQIMIKEKEEMKRVQEALQIERDQLKENTKEIVAKMKESQEKEYQFLKMTAVNETQEKMCEIEHLKEQFETQKLNLENIETENIRLTQILHENLEEMRSVTKERDDLRSVEETLKVERDQLKENLRETITRDLEKQEELKIVHMHLKEHQETIDKLRGIVSEKTNEISNMQKDLEHSNDALKAQDLKIQEELRIAHMHLKEQQETIDKLRGIVSEKTDKLSNMQKDLENSNAKLQEKIQELKANEHQLITLKKDVNETQKKVSEMEQLKKQIKDQSLTLSKLEIENLNLAQELHENLEEMKSVMKERDNLRRVEETLKLERDQLKESLQETKARDLEIQQELKTARMLSKEHKETVDKLREKISEKTIQISDIQKDLDKSKDELQKKDRQNHQVKPEKRLLSDGQQHLMESLREKCSRIKELLKRYSEMDDHYECLNRLSLDLEKEIEFHRIMKKLKYVLSYVTKIKEEQHECINKFEMDFIDEVEKQKELLIKIQHLQQDCDVPSRELRDLKLNQNMDLHIEEILKDFSESEFPSIKTEFQQVLSNRKEMTQFLEEWLNTRFDIEKLKNGIQKENDRICQVNNFFNNRIIAIMNESTEFEERSATISKEWEQDLKSLKEKNEKLFKNYQTLKTSLASGAQVNPTTQDNKNPHVTSRATQLTTEKIRELENSLHEAKESAMHKESKIIKMQKELEVTNDIIAKLQAKVHESNKCLEKTKETIQVLQDKVALGAKPYKEEIEDLKMKLVKIDLEKMKNAKEFEKEISATKATVEYQKEVIRLLRENLRRSQQAQDTSVISEHTDPQPSNKPLTCGGGSGIVQNTKALILKSEHIRLEKEISKLKQQNEQLIKQKNELLSNNQHLSNEVKTWKERTLKREAHKQVTCENSPKSPKVTGTASKKKQITPSQCKERNLQDPVPKESPKSCFFDSRSKSLPSPHPVRYFDNSSLGLCPEVQNAGAESVDSQPGPWHASSGKDVPECKTQ

SEQ ID NO: 12 - DNA, 20 bp, Homo sapiens
acagaaaaaggaccgacaga

SEQ ID NO: 13 - DNA, 20 bp, Homo sapiens
agatcaagagaatgaactca

SEQ ID NO: 14 - DNA, 20 bp, Homo sapiens
agatcaagaggaaagcattg

SEQ ID NO: 15 - protein, 10 aa, Homo sapiens
ELQKKDRQNH

SEQ ID NO: 16 - protein, 10 aa, Homo sapiens
KKDQENELSS

SEQ ID NO: 17 - protein, 10 aa, Homo sapiens
KKDQEESIED

SEQ ID NO: 18 - DNA, 285 bp, Homo sapiens
atccaagaacttcagaaaaaagaacttcaactgcttagagtgaaagaagatgtcaatatgagtcataaaaaaattaatgaaatggaacagttgaagaagcaatttgagccaaactatctatgcaagtgtgagatggataacttccagttgactaagaaacttcatgaaagccttgaagaaataagaattgtagctaaagaaagagatgagctaaggaggataaaagaatctctcaaaatggaaagggaccaattcatagcaaccttaagggaaatgatagctaga

SEQ ID NO: 19 - protein, 95 aa, Homo sapiens
IQELQKKELQLLRVKEDVNMSHKKINEMEQLKKQFEPNYLCKCEMDNFQLTKKLHESLEEIRIVAKERDELRRIKESLKMERDQFIATLREMIAR

SEQ ID NO: 20 - DNA, 28 bp, Homo sapiens
caacaggaactaaaaactgctcgtatgc

SEQ ID NO: 21 - DNA, 28 bp, Homo sapiens
aggctttccataaggtgctgttgtccat

SEQ ID NO: 22 - DNA, 28 bp, Homo sapiens
taacacggatgctggtgacctcttcttc

SEQ ID NO: 23 - DNA, 28 bp, Homo sapiens
aaaggctgattctctcttggcatcaagg

SEQ ID NO: 24 - DNA, 28 bp, Homo sapiens
atggcggaggaaggagccgtggccgtct

SEQ ID NO: 25 - DNA, 28 bp, Homo sapiens
ctactgagttttgcactcaggcacatcc

(19) United States
(12) Patent Application Publication
(10) Pub. No.: US 2005/0003402 A1
(43) Pub. Date: Jan. 6, 2005

(54) Novel isoforms of centromere protein E (CENPE)

(76) Inventors: Christopher D. Armour, Kirkland, WA (US); John C. Castle, Seattle, WA (US); Philip W. Garrett-Engele, Seattle, WA (US); Zhengyan Kan, Bellevue, WA (US); Patrick M. Loerch, Brookline, MA (US); Nicholas F. Tsinoremas, Sammamish, WA (US)

Correspondence Address: R. Douglas Bradley;Rosetta Inpharmatics LLC, Legal Department, 401 Terry Avenue North, Seattle, WA, 98109, US

(21) Appl. No.: 10/828,985

(22) Filed: Apr. 21, 2004

Related U.S. Application Data
(60) Provisional application No. 60/464,905, filed on Apr. 23, 2003.
(60) Provisional application No. 60/510,701, filed on Oct. 10, 2003.

(51) Int. Cl. C12Q 1/68; C12N 9/12; C12N 9/22; C07H 21/04

(52) U.S. Cl. 435/6; 435/69.1; 435/320.1; 435/325; 530/358; 536/23.2; 435/199

(57) ABSTRACT

The present invention features nucleic acids and polypeptides encoding three novel variant isoform of centromere protein E (CENPE). The polynucleotide sequence of CENPEv2, CENPEv3, and CENPEv4 are provided by SEQ ID NO 6, SEQ ID NO 8, and SEQ ID NO 10, respectively. The amino acid sequences for CENPEv2, CENPEv3, and CENPEv4 are provided by SEQ ID NO 7, SEQ ID NO 9, and SEQ ID NO 11, respectively. The present invention also provides methods for using CENPEv2, CENPEv3, and CENPEv4 polynucleotides and proteins to screen for compounds that bind to CENPEv2, CENPEv3, and CENPEv4, respectively. The present invention also provides for methods to detect the presence of cancer and for inhibiting abnormal cell proliferation.

This application claims priority to U.S. Provisional Patent Application Ser. No. 60/464,905 filed on Apr. 23, 2003, and U.S. Provisional Patent Application Ser. No. 60/510,701 filed on Oct. 10, 2003, each of which is incorporated by reference herein in its entirety.

### BACKGROUND OF THE INVENTION

The references cited herein are not admitted to be prior art to the claimed invention.

Mitosis is the process of cell division whereby chromosomes are duplicated and separated into daughter cells. In eukaryotic cells, separation of replicated chromosome pairs (chromatids) is accomplished via a spindle apparatus composed of a network of microtubule fibers emanating from two opposite spindle poles. Sister chromatids are attached to each other via the centromere and are attached to the spindle microtubules via a kinetochore complex associated with the centromere. Spindle microtubules have a defined polarity, with the slow-growing minus end attached to the spindle pole, and the fast-growing plus-end extending into the cytoplasm, ultimately attaching to chromosomes at kinetochores.

During prometaphase, the nuclear envelope dissolves, allowing kinetochores access to the microtubules emanating from the spindle poles. When the plus end of a microtubule comes into contact with one of the kinetochores of a chromosome pair, kinetochore resident binding proteins capture the microtubule and prevent it from depolymerizing. Once a kinetochore is attached to a microtubule, the chromosome moves rapidly toward the attached pole. At this point the chromatid pair is mono-oriented. Eventually the sister kinetochore captures microtubules emanating from the opposite pole and the chromosome becomes bi-oriented.

Once attached to microtubules, chromosomes undergo an oscillatory motion, switching between pole-ward motion and motion away from the pole. As one chromatid moves towards its attached pole, the sister chromatid moves away from its pole. This motion is accomplished via kinetochore motor activity that drives chromosomes toward the pole and polar ejection forces that push chromosomes away from the pole. The oscillatory movement is accompanied by depolymerization or shortening of microtubules at the leading (pole-ward) kinetochore and polymerization or elongation of microtubules at the lagging kinetochore. During congression (the process by which chromosomes move toward the metaphase plate), the time spent moving away from the pole is greater than pole-ward movement, resulting in net movement toward the equator (for a review of kinetochore and spindle interactions during mitosis, see Compton, Duane A., 2000, Ann. Rev. Biochem. 69, 95-114).

Centromere protein E (CENPE) is a protein transiently associated with the kinetochore complex during mitosis. CENPE is a cytoplasmic resident protein during interphase and prophase and does not become bound to the kinetochore until prometaphase, immediately after the breakdown of the nuclear envelope. It remains associated with the centromere during chromosome congression to the metaphase plate and throughout pole-ward segregation during anaphase-A. It gradually relocates to the spindle midzone during anaphase-B, and is degraded at the end of mitosis (Yen, et. al., 1991, EMBO J. 10, 1245-1254; Brown, et. al., 1996, J. Cell Science 109, 961-969).

CENPE is a member of the kinesin super family of molecular motors responsible for trafficking cargo within the cell. Kinesins share an evolutionary conserved catalytic motor domain of 330-340 amino acids that hydrolzes ATP to generate force and movement. The motor domain is attached to an alpha helical coiled-coil stalk domain and a globular tail domain (for review of kinesins, see Goldstein, Lawrence, S. B. and Philip, Alastair V., 1999, Ann. Rev. Cell Dev. Biol. 15, 141-183). CENPE is a 312 kD kinesin-like motor protein. The CENPE amino terminus 335 amino acids share extensive homology with the motor domains of other kinesin family members and contain a 120 amino acid micro-tubule binding sequence highly conserved among kinesins. CENPE also contains an alpha-helical stock and globular tail domain characteristic of kinesins (Yen, et. al., 1992, Nature 359, 536-539). CENPE has a kinetochore binding domain that is in a 350 amino acid region located within the last 540 amino acids of the carboxy-terminus, but is adjacent to and upstream of the carboxy-terminal microtubule binding domain (Chan, et. al., 1998, J. Cell Biol. 143, 49-63). The carboxy terminal microtubule binding domain is not ATP dependent, unlike the amino-terminal microtubule binding domain that is ATP dependent (Zecevic, et. al., 1998, J. Cell Biol. 142, 1547-1558). Binding of microtubules to the CENPE carboxy terminus appears to be dependent on its phosphorylation status, as phosphorylation of CENPE carboxy-terminal sites during mitosis decreases the binding of microtubules to the carboxy terminus (Liao, et. al., 1994, Science 265, 394-398).

CENPE is not critical for the kinetochore to bind microtubules, but is essential to maintain and stabilize kinetochore/microtubule connections. CENPE and its motor domain are essential for both mono-oriented chromosomes to establish bi-polar attachments and for bi-oriented chromosomes to move to and align at the metaphase plate (Schaar, et. al., 1997, J. Cell Biol. 139, 1373-1382). Data show that CENPE is a plus-end directed motor, i.e. moves toward the plus end of microtubules (Wood, et. al., 1997, Cell 91, 357-366).

Once all chromosomes have bi-polar attachments and are aligned at the spindle equator, the cell cycle can progress from metaphase to anaphase, where sister chromatids dissociate and move to opposite spindle poles. Because of the critical importance of proper chromosome segregation during mitosis, progression to anaphase cannot occur until certain requirements are fulfilled. The monitoring of these requirements is accomplished by a spindle assembly checkpoint, also known as a kinetochore dependent checkpoint. This checkpoint prevents the cell from entering into anaphase until all of the sister chromatid pairs are attached to microtubules and tension is created between the sister kinetochores indicating that they are attached to opposite spindle poles and are properly aligned at the equator (for review, see McIntosh, et. al., 2002, Ann. Rev. Cell & Develop. Biol., 18, 193-219).

Studies have shown that CENPE is a crucial component of the kinetochore dependent checkpoint. CENPE is required for both stable kinetochore/microtubule attachments and for creating tension between the sister kinetochores. Absence of CENPE leads to almost total mitotic arrest (Yao, et. al., 2000, Nature Cell Biol. 2, 484-491).

Many human cancers have been linked to chromosomal instability that leads to an abnormal number of chromosomes (aneuploidy) (Lengauer, et. al., 1997, Nature 386, 623-627; Sorger, et. al., 1997, Curr. Op. Cell Biol. 9, 807-814). Mutations in the mitotic checkpoint gene hBUB 1 have been implicated in colon cancers and it has been suggested that other checkpoint genes could be involved in other types of cancers (Cahill, et. al., 1998, Nature 392, 300-303). Drugs that effect kinetochore-microtubule attachments, such as paclitaxel (taxol) and the vinca alkaloids (vinblastine and vincristine), have been shown to be effective chemotherapeutics for cancer treatment. These drugs cause mitotic arrest leading to cell apoptosis (Sorger, et. al., 1997).

It has also been shown that the farnesyl protein transferase inhibitor SCH66336 acts in synergy with and enhances the antitumor activity of taxol (Shi, et. al., 2000, Cancer Chemother. Pharmacol. 46, 387-393). CENPE has a farnesylation site at its extreme carboxy end, and SCH66336 blocks the farnesylation of CENPE, preventing its association with microtubules, and delaying the mitotic process in prometaphase (Ashar, et. al., 2000, J. Biol. Chem. 275, 30451-30457).

Mitotic arrest has also been accomplished by injecting cells with antibodies specific to CENPE. The monoclonal antibody mAB 177, directed to the stalk region of CENPE, when microinjected into human CF-PAC (cystic fibrosis pancreatic cancer) cells, slows or stops the transition from metaphase to anaphase (Yen, et. al., 1991). Yen, et. al. hypothesized that antibodies directed to CENPE delay or stop mitotic progression by either occluding CENPE interaction with other essential components or by blocking a critical CENPE activity. Antibodies directed to the amino or tail end of CENPE slow chromosome motility, while those directed to the neck region, which connects the motor domain to the stalk domain, stop movement completely by dissociating the kinetochore from depolymerizing microtubules (Lombillo, et. al., 1995, J. Cell Biol. 128, 107-115). Antibodies directed to CENPE rod domain (HX-1), or to the carboxy terminus domain (DraB) injected into HeLa cells or U2OS cells prevented chromosomes from aligning at the spindle equator resulting in mitotic arrest and apoptosis. The antibodies prevented CENPE from associating with the kinetochore, either by sterically interfering with its ability to bind to kinetochores or by obscuring the kinetochore-targeting domain from its binding site. Over expression of a CENPE mutant that lacked a motor domain was found to saturate kinetochore binding sites and also prevented chromosome alignment (Schaar, et. al., 1997).

An antisense oligonucleotide centered on the ATG initiation site blocked the synthesis of CENPE and caused mitotic arrest (Yao, et. al., 2000, Nature Cell Biol. 2, 484-491).

Given the demonstrated effectiveness in cancer treatment of drugs that cause mitotic arrest and that inhibition of CENPE causes mitotic arrest as discussed above, CENPE is an important therapeutic target for cancer treatment. CENPE has also been implicated in rheumatic diseases such as systemic sclerosis and rheumatoid arthritis. Autoantibodies to CENPE have been found in patients with systemic sclerosis (Rattner, et. al., 1996, Arthritis Rheum 39, 1355-1361). CENPE mRNA was found to be up-regulated in rheumatoid synovial fibroblasts and may be involved in the pathophysiology of rheumatoid arthritis (Kullmann, et. al., 1999, Arthritis Res. 1, 71-80). Thus, CENPE is also implicated as being a drug target for the treatment of rheumatic disorders.

Because of the multiple therapeutic values of drugs targeting CENPE, there is a need in the art for compounds that selectively bind to isoforms of CENPE. The present invention is directed towards novel CENPE isoforms and uses thereof.

### BRIEF DESCRIPTION OF THE FIGURES

FIG. 1A illustrates the exon structure of CENPE mRNA corresponding to the published reference form variant of CENPE mRNA (labeled CENPEv1 NM_{—}001813.1), and the exon structure corresponding to the inventive variant forms (labeled CENPEv2, CENPEv3, and CENPEv4). FIG. 1B depicts the nucleotide sequences of the exon junctions resulting from the splicing of exon 37 to exon 38, and exon 38 to exon 39 in the case of CENPEv1 mRNA; the splicing of reference CENPEv1 exon 37 to reference CENPEv1 exon 39 in the case of CENPEv2 mRNA; the splicing of exon 16 to exon 18 in the case of CENPEv3 mRNA; and the splicing of exon 16 to exon 19 in the case of CENPEv4 mRNA. In FIG. 1B, in the case of CENPEv2, the nucleotides shown in italics represent the 20 nucleotides at the 3′ end of exon 37 and the nucleotides shown in underline represent the 20 nucleotides at the 5′ end of exon 39; in the case of CENPEv3, the nucleotides shown in italics represent the 20 nucleotides at the 3′ end of exon 16 and the nucleotides shown in underline represent the 20 nucleotides at the 5′ end of exon 18; in the case of CENPEv4, the nucleotides shown in italics represent the 20 nucleotides at the 3′ end of exon 16 and the nucleotides shown in underline represent the 20 nucleotides at the 5′ end of exon 19.

### SUMMARY OF THE INVENTION

Microarray experiments and RT-PCR have been used to identify and confirm the presence of novel variants of human CENPE mRNA. More specifically, the present invention features polynucleotides encoding novel protein isoforms of CENPE. One such novel protein isoform, herein referred to as CENPE variant 2 (CENPEv2), is the prevalent isoform expressed in normal tissue. A polynucleotide sequence encoding CENPEv2 is provided by SEQ ID NO 6. An amino acid sequence for CENPEv2 is provided by SEQ ID NO 7. A polynucleotide sequence encoding CENPEv3 is provided by SEQ ID NO 8. An amino acid sequence for CENPEv3 is provided by SEQ ID NO 9. A polynucleotide sequence encoding CENPEv4 is provided by SEQ ID NO 10. An amino acid sequence for CENPEv4 is provided by SEQ ID NO 11.

Thus, a first aspect of the present invention describes a purified CENPEv2 encoding nucleic acid, a purified CENPEv3 encoding nucleic acid, and a purified CENPEv4 encoding nucleic acid. The CENPEv2 encoding nucleic acid comprises SEQ ID NO 6 or the complement thereof. The CENPEv3 encoding nucleic acid comprises SEQ ID NO 8 or the complement thereof. The CENPEv4 encoding nucleic acid comprises SEQ ID NO 10 or the complement thereof. Reference to the presence of one region does not indicate that another region is not present. For example, in different embodiments the inventive nucleic acid can comprise, consist, or consist essentially of an encoding nucleic acid sequence of SEQ ID NO 6, can comprise, consist, or consist essentially of an encoding nucleic acid sequence of SEQ ID NO 8, or alternatively, can comprise, consist, or consist essentially of an encoding nucleic acid sequence of SEQ ID NO 10.

Another aspect of the present invention describes a purified CENPEv2 polypeptide that can comprise, consist or consist essentially of the amino acid sequence of SEQ ID NO 7. An additional aspect describes a purified CENPEv3 polypeptide that can comprise, consist or consist essentially of the amino acid sequence of SEQ ID NO 9. An additional aspect describes a purified CENPEv4 polypeptide that can comprise, consist or consist essentially of the amino acid sequence of SEQ ID NO 11.

Another aspect of the present invention describes expression vectors. In one embodiment of the invention, the inventive expression vector comprises a nucleotide sequence encoding a polypeptide comprising, consisting, or consisting essentially of SEQ ID NO 7, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 9, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 11, wherein the nucleotide sequence is transcriptionally coupled to an exogenous promoter.

Alternatively, the nucleotide sequence comprises, consists, or consists essentially of SEQ ID NO 6, and is transcriptionally coupled to an exogenous promoter. In another embodiment, the nucleotide sequence comprises, consists, or consists essentially of SEQ ID NO 8, and is transcriptionally coupled to an exogenous promoter. In another embodiment, the nucleotide sequence comprises, consists, or consists essentially of SEQ ID NO 10, and is transcriptionally coupled to an exogenous promoter

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 6, SEQ ID NO 8, or SEQ ID NO 10, or a nucleotide sequence encoding a polypeptide comprising, consisting, or consisting essentially of an amino acid sequence of SEQ ID NO 7, SEQ ID NO 9, or SEQ ID NO 11, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing CENPEv2, CENPEv3, or CENPEv4 polypeptide comprising SEQ ID NO 7, SEQ ID NO 9, or SEQ ID NO 11, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to CENPEv2 as compared to one or more CENPE isoform polypeptides that are not CENPEv2. In another embodiment, a purified antibody preparation is provided comprising an antibody that binds selectively to CENPEv3 as compared to one or more CENPE isoform polypeptides that are not CENPEv3. In another embodiment, a purified antibody preparation is provided comprising an antibody that binds selectively to CENPEv4 as compared to one or more CENPE isoform polypeptides that are not CENPEv4.

Another aspect of the present invention provides a method of screening for a compound that binds to CENPEv2, CENPEv3, CENPEv4, or fragments thereof. In one embodiment, the method comprises the steps of: (a) expressing a polypeptide comprising the amino acid sequence of SEQ ID NO 7 or a fragment thereof from recombinant nucleic acid; (b) providing to said polypeptide a labeled CENPE ligand that binds to said polypeptide and a test preparation comprising one or more test compounds; (c) and measuring the effect of said test preparation on binding of said test preparation to said polypeptide comprising SEQ ID NO 7. Alternatively, this method could be performed using SEQ ID NO 9 or SEQ ID NO 11, instead of SEQ ID NO 7.

In another embodiment of the method, a compound is identified that binds selectively to CENPEv2 polypeptide as compared to one or more CENPE isoform polypeptides that are not CENPEv2. This method comprises the steps of: providing a CENPEv2 polypeptide comprising SEQ ID NO 7; providing a CENPE isoform polypeptide that is not CENPEv2; contacting said CENPEv2 polypeptide and said CENPE isoform polypeptide that is not CENPEv2 with a test preparation comprising one or more test compounds; and determining the binding of said test preparation to said CENPEv2 polypeptide and to CENPE isoform polypeptide that is not CENPEv2, wherein a test preparation that binds to said CENPEv2 polypeptide but does not bind to said CENPE isoform polypeptide that is not CENPEv2 contains a compound that selectively binds said CENPEv2 polypeptide. Alternatively, the same method can be performed using CENPEv3 polypeptide comprising, consisting, or consisting essentially of SEQ ID NO 9. Alternatively, the same method can be performed using CENPEv4 polypeptide comprising, consisting, or consisting essentially of SEQ ID NO 11.

In another embodiment of the invention, a method is provided for screening for a compound able to bind to or interact with a CENPEv2 protein or a fragment thereof comprising the steps of: expressing a CENPEv2 polypeptide comprising SEQ ID NO 7 or a fragment thereof from a recombinant nucleic acid; providing to said polypeptide a labeled CENPE ligand that binds to said polypeptide and a test preparation comprising one or more compounds; and measuring the effect of said test preparation on binding of said labeled CENPE ligand to said polypeptide, wherein a test preparation that alters the binding of said labeled CENPE ligand to said polypeptide contains a compound that binds to or interacts with said polypeptide. In an alternative embodiment, the method is performed using CENPEv3 polypeptide comprising, consisting, or consisting essentially of SEQ ID NO 9, or a fragment thereof. In an alternative embodiment, the method is performed using CENPEv4 polypeptide comprising, consisting, or consisting essentially of SEQ ID NO 11, or a fragment thereof.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### Definitions

Unless defined otherwise, all technical and scientific terms used herein have the meaning commonly understood by one of ordinary skill in the art to which this invention belongs.

As used herein, “CENPE” refers to a centromeric protein E that has a published genomic sequence accession number of NT_{—}006383.13. In contrast, reference to a CENPE isoform includes a published variant, NP_{—}001804.1, and other polypeptide isoform variants of CENPE.

As used herein, “CENPEv1” refers to a published variant isoform of human CENPE protein, NP_{—}001804.1.

As used herein, “CENPEv2”, refers to a variant isoform of human CENPE protein, wherein the variant is the isoform prevalently expressed in normal tissue and has the amino acid sequence set forth in SEQ ID NO 7.

As used herein, “CENPEv3” and “CENPEv4” refer to variant isoforms of human CENPE protein, wherein the variants have the amino acid sequence set forth in SEQ ID NO 9 (for CENPEv3) and SEQ ID NO 11 (for CENPEv4).

As used herein, “CENPE” refers to polynucleotides encoding CENPE.

As used herein, “CENPEv1” refers to polynucleotides encoding CENPEv1 having an amino acid sequence published as NP_{—}001804.1.

As used herein, “CENPEv2” refers to polynucleotides encoding CENPEv2 having an amino acid sequence set forth in SEQ ID NO 7.

As used herein, “CENPEv3” refers to polynucleotides encoding CENPEv3 having an amino acid sequence set forth in SEQ ID NO 9.

As used herein, “CENPEv4” refers to polynucleotides encoding CENPEv4 having an amino acid sequence set forth in SEQ ID NO 11.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in an isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human CENPEv2, CENPEv3, and CENPEv4 that are alternatively spliced isoforms of CENPE, and to the amino acid sequences encoding this protein. Surprisingly, CENPEv2 has been found by the inventors to represent the CENPE isoform that is most prevalently expressed in normal tissue (see Example 2). The nucleic acid CENPEv1 published reference sequence NM_{—}001813.1 encoding CENPEv1 protein NP_{—}001804.1, also reported in U.S. Pat. No. 6,544,766, was originally detected in a human breast cancer cell line, ATCC CRL 1500. The novel variant described herein, CENPEv2, was detected in 39 normal tissue samples as well as in four cancer cell lines assayed. The reference CENPEv1 isoform was only detected at high levels in one tissue, and was weakly detected in a small number of other tissues assayed. SEQ ID NO 6, SEQ ID NO 8, and SEQ ID NO 10 are polynucleotide sequences representing exemplary open reading frame that encode the CENPEv2, CENPEv3, and CENPEv4 proteins, respectively.

The novel CENPEv2 can be distinguished from the published reference CENPEv1 (NP_{—}1804) based upon the presence at position 300 of an alanine amino acid residue (CENPEv2) instead of proline (CENPEv1); and the absence in CENPEv2 of amino acids at positions 1972 through 2066 of CENPEv1. Amino acids are numbered counting the initiation methionine as occupying position one. CENPEv1 and CENPEv2 mRNAs differ based upon the alternative splicing of intron 37 sequence. In particular, CENPEv1 mRNA includes a region of intron 37 sequence as an additional exon, referred to as “exon 38.” CENPEv2 mRNA does not contain the published “exon 38” sequence.

CENPEv2, CENPEv3, and CENPEv4 polynucleotide sequences encoding CENPEv2, CENPEv3, and CENPEv4 proteins, respectively, as exemplified and enabled herein, include a number of specific, substantial and credible utilities. For example, CENPEv2, CENPEv3, and CENPEv4 encoding nucleic acids were identified in a mRNA sample obtained from a human source (see Example 1). Such nucleic acids can be used as hybridization probes to distinguish between cells that produce CENPEv2, CENPEv3, and CENPEv4 transcripts from human or non-human cells (including bacteria) that do not produce such transcripts. Furthermore, due to the fact that CENPEv2 mRNA does not contain the region of intron 37 that is designated in CENPEv1 as representing exon 38 coding sequence, the presence of CENPEv1 exon 38 coding sequence can be used as a screen for the detection of cancer; i.e., the CENPEv1 exon 38 encoding nucleic acids can be used as hybridization probes to detect the presence of CENPEv1 exon 38 in cells that may be cancerous, in particular breast cancer. Similarly, antibodies specific for CENPEv2, CENPEv3, or CENPEv4 can be used to distinguish between cells that express CENPEv2, CENPEv3, or CENPEv4 from human or non-human cells (including bacteria) that do not express CENPEv2, CENPEv3, or CENPEv4. Also, antibodies specific for the polypeptide region encoded by CENPEv1 exon 38 can also be used to detect the presence of CENPEv1 in cells that may be cancerous.

Drugs that cause mitotic arrest and subsequent cell death have proven to be effective cancer therapeutics (Sorger, et. al. 1997). A number of studies have demonstrated that inhibition of CENPE can cause mitotic arrest (Ashar, et. al., 2000; Shi, et. al., 2000; Schaar, et. al., 1997). It is therefore reasonable to assume that modulating CENPE activity could be an effective chemotherapy. CENPE has also been implicated in the pathophysiology of rheumatoid arthritis (Kullmann, et. al., 1999) and thus may be an effective drug target for the treatment of rheumatic diseases. Given the potential importance of CENPE activity to the therapeutic management of cancer and rheumatic diseases, it is of value to identify CENPE isoforms and identify CENPE-ligand compounds that are isoform specific, as well as compounds that are effective ligands for two or more different CENPE isoforms. In particular, it may be important to identify compounds that are effective inhibitors of a specific CENPE isoform activity, yet do not bind to or interact with a plurality of different CENPE isoforms. Compounds that bind to or interact with multiple CENPE isoforms may require higher drug doses to saturate multiple CENPE-isoform binding sites and thereby result in a greater likelihood of secondary non-therapeutic side effects. Furthermore, biological effects could also be caused by the interactions of a drug with the CENPEv2, CENPEv3, or CENPEv4 isoforms specifically. For the foregoing reasons, CENPEv2, CENPEv3, and CENPEv4 proteins represent useful compound binding targets and have utility in the identification of new CENPE-ligands exhibiting a preferred specificity profile and having greater efficacy for their intended use.

In some embodiments, CENPEv2, CENPEv3, and CENPEv4 activity is modulated by a ligand compound to achieve one or more of the following: prevent or reduce the risk of occurrence, or recurrence of cancer, rheumatoid arthritis, and systemic sclerosis. Compounds that treat cancers are particularly important because of the cause-and-effect relationship between cancers and mortality (National Cancer Institute's Cancer Mortality Rates Registry, http://www3.cancer.gov/atlasplus/charts.html, last visited Dec. 31, 2002).

Compounds modulating CENPEv2, CENPEv3, or CENPEv4 include agonists, antagonists, and allosteric modulators. Inhibitors of CENPE may achieve clinical efficacy by a number of known or unknown mechanisms. While not wishing to be limited to any particular theory of therapeutic efficacy, generally, but not always, CENPEv2, CENPEv3, or CENPEv4 compounds will be used to inhibit binding of CENPE to the kinetochore or to microtubules to cause mitotic delay and apoptosis (Ashar, et. al., 2000; Schaar, et. al., 1997; Lombillo, et. al., 1995; Yen, et. al., 1991).

CENPEv2, CENPEv3, and CENPEv4 activity can also be affected by modulating the cellular abundance of transcripts encoding CENPEv2, CENPEv3, or CENPEv4, respectively. Compounds modulating the abundance of transcripts encoding CENPEv2, CENPEv3, or CENPEv4 include a cloned polynucleotide encoding CENPEv2, CENPEv3, or CENPEv4, respectively, that can express CENPEv2, CENPEv3, or CENPEv4 in vivo, antisense nucleic acids targeted to CENPEv2, CENPEv3, or CENPEv4 transcripts, and enzymatic nucleic acids, such as ribozymes and RNAi, targeted to CENPEv2, CENPEv3, or CENPEv4 transcripts.

In some embodiments, CENPEv2, CENPEv3, or CENPEv4 activity is modulated to achieve a therapeutic effect upon diseases in which regulation of mitosis is desirable. For example, various cancers may be treated by inhibiting the binding of CENPE to the kinetochore or the microtubules to cause mitotic arrest and apoptosis. In other embodiments, rheumatic diseases may be treated by modulating CENPEv2, CENPEv3, or CENPEv4 activity to affect rheumatoid pathophysiology.

CENPEv2, CENPEv3, and CENPEv4 Nucleic Acids

CENPEv2 nucleic acids contain regions that encode for polypeptides comprising, consisting, or consisting essentially of SEQ ID NO 7. CENPEv3 nucleic acids contain regions that encode for polypeptides comprising, consisting, or consisting essentially of SEQ ID NO 9. CENPEv4 nucleic acids contain regions that encode for polypeptides comprising, consisting, or consisting essentially of SEQ ID NO 11. The CENPEv2, CENPEv3, and CENPEv4 nucleic acids have a variety of uses, such as use as a hybridization probe or PCR primer to identify the presence of CENPEv2, CENPEv3, or CENPEv4 nucleic acids, respectively; use as a hybridization probe or PCR primer to identify nucleic acids encoding for proteins related to CENPEv2, CENPEv3, or CENPEv4, respectively; and/or use for recombinant expression of CENPEv2, CENPEv3, or CENPEv4 polypeptides, respectively. In particular, CENPEv2, CENPEVv3, or CENPEv4 polynucleotides do not have the polynucleotide region that comprises exon 38 of the CENPEv1 gene. In particular, CENPEv3 polynucleotides do not have the polynucleotide region that comprises exon 17 of the CENPE gene. CENPEv4 polynucleotides do not have the polynucleotide region that comprises exon 17 and exon 18 of the CENPE gene.

Regions in CENPEv2, CENPEv3, or CENPEv4 nucleic acid that do not encode for CENPEv2, CENPEv3, or CENPEv4, or are not found in SEQ ID NO 6, SEQ ID NO 8, or SEQ ID NO 10, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding CENPEv2, CENPEv3, or CENPEv4 related proteins from different sources. Obtaining nucleic acid CENPEv2, CENPEv3, or CENPEv4 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

CENPEv2, CENPEv3, or CENPEv4 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CENPEv2, CENPEv3, or CENPEv4 Probes

Probes for CENPEv2, CENPEv3, or CENPEv4 contain a region that can specifically hybridize to CENPEv2, CENPEv3, or CENPEv4 target nucleic acids, respectively, under appropriate hybridization conditions and can distinguish CENPEv2, CENPEv3, or CENPEv4 nucleic acids from each other and from non-target nucleic acids, in particular polynucleotides containing CENPEv1 exon 38 and CENPE polynucleotides containing exons 17 and 18. Probes for CENPEv2, CENPEv3, or CENPEv4 can also contain nucleic acid regions that are not complementary to CENPEv2, CENPEv3, or CENPEv4 nucleic acids.

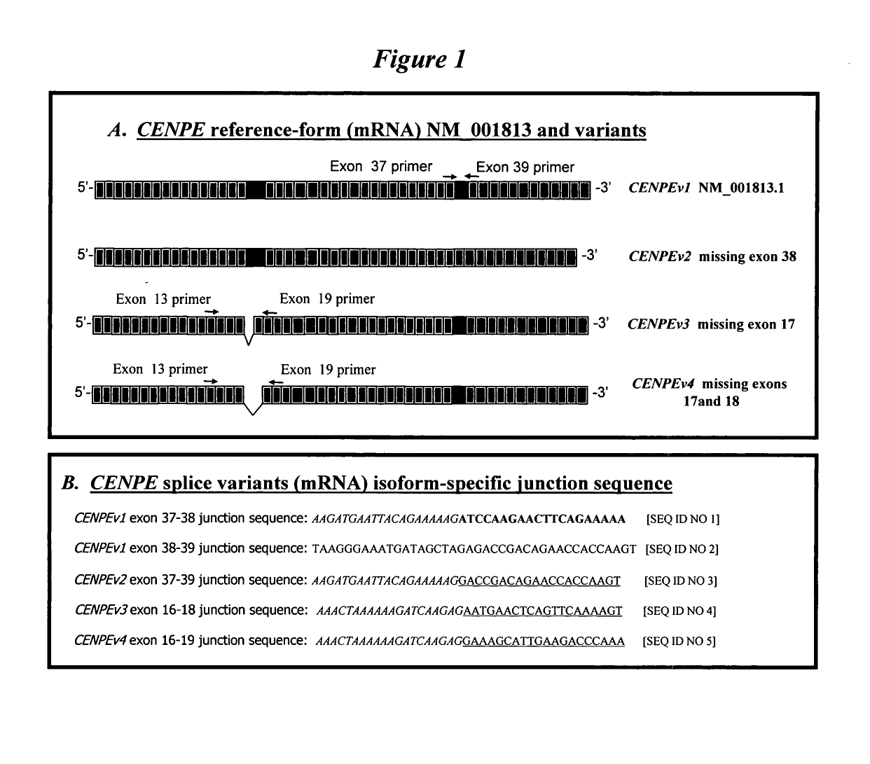

In embodiments where, for example, CENPEv2, CENPEv3, or CENPEv4 polynucleotide probes are used in hybridization assays to specifically detect the presence of CENPEv2, CENPEv3, or CENPEv4 polynucleotides in samples, the CENPEv2, CENPEv3, or CENPEv4 polynucleotides comprise at least 20 nucleotides of the CENPEv2, CENPEv3, or CENPEv4 sequence that correspond to the respective novel exon junction polynucleotide regions. In particular, for detection of CENPEv2, CENPEv3, or CENPEv4, the probe comprises at least 20 nucleotides of the CENPEv2, CENPEv3, or CENPEv4 sequence that corresponds to an exon junction polynucleotide created by the alternative splicing of exon 37 to exon 39 of the CENPEv1 transcript (see FIGS. 1A and 1B). For example, the polynucleotide sequence: 5′ ACAGAAAAAGGACCGACAGA 3′ [SEQ ID NO 12] represents one embodiment of such an inventive CENPEv2, CENPEv3, or CENPEv4 polynucleotide wherein a first 10 nucleotides region is complementary and hybridizable to the 3′ end of CENPEv1 exon 37 and a second 10 nucleotide region is complementary and hybridizable to the 5′ end of CENPEv1 exon 39.

In another embodiment, for detection of CENPEv3, the probe comprises at least 20 nucleotides of the CENPEv3 sequence that corresponds to an exon junction polynucleotide created by the alternative splicing of exon 16 to exon 18 of the CENPE transcript (see FIGS. 1A and 1B). For example, the polynucleotide sequence: 5′ AGATCAAGAGAATG AACTCA 3′ [SEQ ID NO 13] represents one embodiment of such an inventive CENPEv3 polynucleotide wherein a first 10 nucleotides region is complementary and hybridizable to the 3′ end of exon 16 and a second 10 nucleotide region is complementary and hybridizable to the 5′ end of exon 18.

In another embodiment, for detection of CENPEv4, the probe comprises at least nucleotides of the CENPEv4 sequence that corresponds to an exon junction polynucleotide created by the alternative splicing of exon 16 to exon 19 of the CENPE transcript (see FIGS. 1A and 1B). For example, the polynucleotide sequence: 5′ AGATCAAGAGGAAAG CATTG 3′ [SEQ ID NO 14] represents one embodiment of such an inventive CENPEv4 polynucleotide wherein a first 10 nucleotides region is complementary and hybridizable to the 3′ end of exon 16 and a second 10 nucleotide region is complementary and hybridizable to the 5′ end of exon 19.

In some embodiments, the first 20 nucleotides of a CENPEv2, CENPEv3, or CENPEv4 probe comprise a first continuous region of 5 to 15 nucleotides that is complementary and hybridizable to the 3′ end of CENPEv1 exon 37 and a second continuous region of 5 to nucleotides that is complementary and hybridizable to the 5′ end of CENPEv1 exon 39. In some embodiments, the first 20 nucleotides of a CENPEv3 probe comprise a first continuous region of 5 to 15 nucleotides that is complementary and hybridizable to the 3′ end of exon 16 and a second continuous region of 5 to 15 nucleotides that is complementary and hybridizable to the 5′ end of exon 18. In some embodiments, the first 20 nucleotides of a CENPEv4 probe comprise a first continuous region of 5 to 15 nucleotides that is complementary and hybridizable to the 3′ end of exon 16 and a second continuous region of 5 to 15 nucleotides that is complementary and hybridizable to the 5′ end of exon 19.

In other embodiments, the CENPEv2, CENPEv3, or CENPEv4 polynucleotides comprise at least 40, 60, 80 or 100 nucleotides of the CENPEv2, CENPEv3, or CENPEv4 sequence, respectively, that correspond to a junction polynucleotide region created by the alternative splicing of CENPEv1 exon 37 to CENPEv1 exon 39 in the case of CENPEv2, CENPEv3, or CENPEv4; that correspond to a junction polynucleotide region created by the alternative splicing of exon 16 to exon 18 in the case of CENPEv3; or in the case of CENPEv4, by the alternative splicing of exon 16 to exon 19 of the primary transcript of the CENPE gene. In embodiments involving CENPEv2, CENPEv3, or CENPEv4, the CENPEv2, CENPEv3, or CENPEv4 polynucleotide is selected to comprise a first continuous region of at least 5 to nucleotides that is complementary and hybridizable to the 3′ end of CENPEv1 exon 37 and a second continuous region of at least 5 to 15 nucleotides that is complementary and hybridizable to the 5′ end of CENPEv1 exon 39. Similarly, in embodiments involving CENPEv3, the CENPEv3 polynucleotide is selected to comprise a first continuous region of at least 5 to nucleotides that is complementary and hybridizable to the 3′ end of exon 16 and a second continuous region of at least 5 to 15 nucleotides that is complementary and hybridizable to the 5′ end of exon 18. Similarly, in embodiments involving CENPEv4, the CENPEv4 polynucleotide is selected to comprise a first continuous region of at least 5 to 15 nucleotides that is complementary and hybridizable to the 3′ end of exon 16 and a second continuous region of at least 5 to 15 nucleotides that is complementary and hybridizable to the 5′ end of exon 19. As will be apparent to a person of skill in the art, a large number of different polynucleotide sequences from the region of the CENPEv1 exon 37 to exon 39 splice junction, the exon 16 to exon 18 splice junction, and the exon 16 to exon 19 splice junction may be selected which will, under appropriate hybridization conditions, have the capacity to detectably hybridize to CENPEv2, CENPEv3, or CENPEv4, respectively, and yet will hybridize to a much less extent or not at all to CENPE isoform polynucleotides wherein CENPEv1 exon 37 is not spliced to CENPEv1 exon 39, wherein exon 16 is not spliced to exon 18, or wherein exon 16 is not spliced to exon 19, respectively.

Preferably, non-complementary nucleic acid that is present has a particular purpose such as being a reporter sequence or being a capture sequence. However, additional nucleic acid need not have a particular purpose as long as the additional nucleic acid does not prevent the CENPEv2, CENPEv3, or CENPEv4 nucleic acid from distinguishing between target polynucleotides, e.g., CENPEv2, CENPEv3, or CENPEv4 polynucleotides, and non-target polynucleotides, including, but not limited to CENPE polynucleotides not comprising the CENPEv1 exon 37 to exon 39 splice junction, the exon 16 to exon 18 junction, or the exon 16 to exon 19 splice junction found in CENPEv2, CENPEv3, or CENPEv4, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5×Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5×Denhardt's solution, 50% formamide at 42° C. for 12 to 48 hours or a washing step carried out in 0.2×SSPE, 0.2% SDS at 65° C. for 30 to 60 minutes.

Recombinant Expression

CENPEv2, CENPEv3, or CENPEv4 polynucleotides, such as those comprising SEQ ID NO 6, SEQ ID NO 8, or SEQ ID NO 10, respectively, can be used to make CENPEv2, CENPEv3, or CENPEv4 polypeptides, respectively. In particular, CENPEv2, CENPEv3, or CENPEv4 polypeptides can be expressed from recombinant nucleic acids in a suitable host or in vitro using a translation system. Recombinantly expressed CENPEv2, CENPEv3, or CENPEv4 polypeptides can be used, for example, in assays to screen for compounds that bind CENPEv2, CENPEv3, or CENPEv4, respectively. Alternatively, CENPEv2, CENPEv3, or CENPEv4 polypeptides can also be used to screen for compounds that bind to one or more CENPE isoforms, but do not bind to CENPEv2, CENPEv3, or CENPEv4, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLac1 (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen) and pYES2 (Invitrogen), Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 6, SEQ ID NO 8, or SEQ ID NO 10 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

CENPEv2, CENPEv3, and CENPEv4 Polypeptides

CENPEv2 polypeptides contain an amino acid sequence comprising, consisting or consisting essentially of SEQ ID NO 7. CENPEv3 polypeptides contain an amino acid sequence comprising, consisting or consisting essentially of SEQ ID NO 9. CENPEv4 polypeptides contain an amino acid sequence comprising, consisting or consisting essentially of SEQ ID NO 11. CENPEv2, CENPEv3, or CENPEv4 polypeptides have a variety of uses, such as providing a marker for the presence of CENPEv2, CENPEv3, or CENPEv4, respectively; use as an immunogen to produce antibodies binding to CENPEv2, CENPEv3, or CENPEv4, respectively; use as a target to identify compounds binding selectively to CENPEv2, CENPEv3, or CENPEv4, respectively; or use in an assay to identify compounds that bind to one or more iosforms of CENPE but do not bind to or interact with CENPEv2, CENPEv3, or CENPEv4, respectively.

In chimeric polypeptides containing one or more regions from CENPEv2, CENPEv3, or CENPEv4 and one or more regions not from CENPEv2, CENPEv3, or CENPEv4, respectively, the region(s) not from CENPEv2, CENPEv3, or CENPEv4, respectively, can be used, for example, to achieve a particular purpose or to produce a polypeptide that can substitute for CENPEv2, CENPEv3, or CENPEv4, or fragments thereof. Particular purposes that can be achieved using chimeric CENPEv2, CENPEv3, or CENPEv4 polypeptides include providing a marker for CENPEv2, CENPEv3, or CENPEv4 activity, respectively, enhancing an immune response, and modulating the progression of mitosis.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CENPEv2, CENPEv3, and CENPEv4

Functional CENPEv2, CENPEv3, and CENPEv4 are different protein isoforms of CENPE. The identification of the amino acid and nucleic acid sequences of CENPEv2, CENPEv3, or CENPEv4 provide tools for obtaining functional proteins related to CENPEv2, CENPEv3, or CENPEv4, respectively, from other sources, for producing CENPEv2, CENPEv3, or CENPEv4 chimeric proteins, and for producing functional derivatives of SEQ ID NO 7, SEQ ID NO 9, or SEQ ID NO 11.

CENPEv2, CENPEv3, or CENPEv4 polypeptides can be readily identified and obtained based on their sequence similarity to CENPEv2 (SEQ ID NO 7), CENPEv3 (SEQ ID NO 9), or CENPEv4 (SEQ ID NO 11), respectively. In particular, CENPEv2, CENPEv3, or CENPEv4 contain an alanine at position 300 and lack the amino acids encoded by exon 38 of CENPEv1; CENPEv3 lacks the amino acids encoded by exon 17 of the CENPE gene, and CENPEv4 lacks the amino acids encoded by exon 17 and exon 18 of the CENPE gene.

Both the amino acid and nucleic acid sequences of CENPEv2, CENPEv3, or CENPEv4 can be used to help identify and obtain CENPEv2, CENPEv3, or CENPEv4 polypeptides, respectively. For example, SEQ ID NO 6 can be used to produce degenerative nucleic acid probes or primers for identifying and cloning nucleic acid polynucleotides encoding for a CENPEv2 polypeptide. In addition, polynucleotides comprising, consisting, or consisting essentially of SEQ ID NO 6 or fragments thereof, can be used under conditions of moderate stringency to identify and clone nucleic acids encoding CENPEv2 polypeptides from a variety of different organisms. The same methods can also be performed with polynucleotides comprising, consisting, or consisting essentially of SEQ ID NO 8 or SEQ ID NO 10, or fragments thereof, to identify and clone nucleic acids encoding CENPEv3 and CENPEv4, respectively.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with CENPEv2, CENPEv3, or CENPEv4 obtained from a particular source, derivatives can be produced. Such derivatives include polypeptides with amino acid substitutions, additions and deletions. Changes to CENPEv2, CENPEv3, or CENPEv4 to produce a derivative having essentially the same properties should be made in a manner not altering the tertiary structure of CENPEv2, CENPEv3, or CENPEv4, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide than glutamate because of its long aliphatic side chain (See, Ausubel, Current Protocols in Molecular Biology, John Wiley, 1987-1998, Supplement 33 Appendix 1C).

CENPEv2, CENPEv3, and CENPEv4 Antibodies

Antibodies recognizing CENPEv2, CENPEv3, or CENPEv4 can be produced using a polypeptide containing SEQ ID NO 7 in the case of CENPEv2, SEQ ID NO 9 in the case of CENPEv3, or SEQ ID NO 11 in the case of CENPEv4, respectively, or a fragment thereof, as an immunogen. Preferably, a CENPEv2 polypeptide used as an immunogen consists of a polypeptide of SEQ ID NO 7 or a SEQ ID NO 7 fragment having at least 10 contiguous amino acids in length corresponding to the polynucleotide region representing the junction resulting from the splicing of exon 37 to exon 39 of the CENPEv1 transcript. Preferably, a CENPEv3 polypeptide used as an immunogen consists of a polypeptide of SEQ ID NO 9 or a SEQ ID NO 9 fragment having at least 10 contiguous amino acids in length corresponding to the polynucleotide region representing the junction resulting from the splicing of exon 16 to exon 18 of the CENPE transcript. Preferably, a CENPEv4 polypeptide used as an immunogen consists of a polypeptide of SEQ ID NO 11 or a SEQ ID NO 11 fragment having at least 10 contiguous amino acids in length corresponding to the polynucleotide region representing the junction resulting from the splicing of exon 16 to exon 19 of the CENPE transcript.

In some embodiments where, for example, CENPEv2 polypeptides are used to develop antibodies that bind specifically to CENPEv2 and not to CENPEv1, the CENPEv2 polypeptides comprise at least 10 amino acids of the CENPEv2 polypeptide sequence corresponding to a junction polynucleotide region created by the alternative splicing of exon 37 to exon 39 of CENPEv1 (see FIG. 1). For example, the amino acid sequence: amino terminus-ELQKKDRQNH-carboxy terminus [SEQ ID NO 15] represents one embodiment of such an inventive CENPEv2 polypeptide wherein a first 5 amino acid region is encoded by nucleotide sequence at the 3′ end of CENPEv1 exon 37 and a second 5 amino acid region is encoded by the nucleotide sequence directly after the novel splice junction. Preferably, at least 10 amino acids of the CENPEv2 polypeptide comprises a first continuous region of 2 to 8 amino acids that is encoded by nucleotides at the 3′ end of CENPEv1 exon 37 and a second continuous region of 2 to 8 amino acids that is encoded by nucleotides at the 5′ end of CENPEv1 exon 39.

In other embodiments where, for example, CENPEv3 polypeptides are used to develop antibodies that bind specifically to CENPEv3 and not to other isoforms of CENPE, the CENPEv3 polypeptides comprise at least 10 amino acids of the CENPEv3 polypeptide sequence corresponding to a junction polynucleotide region created by the alternative splicing of exon 16 to exon 18 of CENPE (see FIG. 1). For example, the amino acid sequence: amino terminus-KKDQENELSS-carboxy terminus [SEQ ID NO 16] represents one embodiment of such an inventive CENPEv3 polypeptide wherein a first 5 amino acid region is encoded by nucleotide sequence at the 3′ end of CENPE exon 16 and a second 5 amino acid region is encoded by the nucleotide sequence directly after the novel splice junction. Preferably, at least 10 amino acids of the CENPEv3 polypeptide comprises a first continuous region of 2 to 8 amino acids that is encoded by nucleotides at the 3′ end of CENPE exon 16 and a second continuous region of 2 to 8 amino acids that is encoded by nucleotides at the 5′ end of CENPE exon 18.

In other embodiments where, for example, CENPEv4 polypeptides are used to develop antibodies that bind specifically to CENPEv4 and not to other isoforms of CENPE, the CENPEv4 polypeptides comprise at least 10 amino acids of the CENPEv4 polypeptide sequence corresponding to a junction polynucleotide region created by the alternative splicing of exon 16 to exon 19 of CENPE (see FIG. 1). For example, the amino acid sequence: amino terminus-KKDQEESIED-carboxy terminus [SEQ ID NO 17] represents one embodiment of such an inventive CENPEv4 polypeptide wherein a first 5 amino acid region is encoded by nucleotide sequence at the 3′ end of CENPE exon 16 and a second 5 amino acid region is encoded by the nucleotide sequence directly after the novel splice junction. Preferably, at least 10 amino acids of the CENPEv4 polypeptide comprises a first continuous region of 2 to 8 amino acids that is encoded by nucleotides at the 3′ end of CENPE exon 16 and a second continuous region of 2 to 8 amino acids that is encoded by nucleotides at the 5′ end of CENPE exon 19.

In other embodiments, CENPEv2-specific antibodies are made using an CENPEv2 polypeptide that comprises at least 20, 30, 40 or 50 amino acids of the CENPEv2 sequence that corresponds to a junction polynucleotide region created by the alternative splicing of CENPEv1 exon 37 to CENPEv1 exon 39. In each case the CENPEv2 polypeptides are selected to comprise a first continuous region of at least 5 to 15 amino acids that is encoded by nucleotides at the 3′ end of CENPEv1 exon 37 and a second continuous region of 5 to 15 amino acids that is encoded by nucleotides directly after the novel splice junction.

In other embodiments, CENPEv3-specific antibodies are made using an CENPEv3 polypeptide that comprises at least 20, 30, 40 or 50 amino acids of the CENPEv3 sequence that corresponds to a junction polynucleotide region created by the alternative splicing of exon 16 to exon 18 of the primary transcript of the CENPE gene. In each case the CENPEv3 polypeptides are selected to comprise a first continuous region of at least 5 to 15 amino acids that is encoded by nucleotides at the 3′ end of exon 16 and a second continuous region of 5 to amino acids that is encoded by nucleotides directly after the novel splice junction.

In other embodiments, CENPEv4-specific antibodies are made using an CENPEv4 polypeptide that comprises at least 20, 30, 40 or 50 amino acids of the CENPEv4 sequence that corresponds to a junction polynucleotide region created by the alternative splicing of exon 16 to exon 19 of the primary transcript of the CENPE gene. In each case the CENPEv4 polypeptides are selected to comprise a first continuous region of at least 5 to 15 amino acids that is encoded by nucleotides at the 3′ end of exon 16 and a second continuous region of 5 to amino acids that is encoded by nucleotides directly after the novel splice junction.

Antibodies to CENPEv2, CENPEv3, or CENPEv4 have different uses, such as to identify the presence of CENPEv2, CENPEv3, or CENPEv4, respectively, and to isolate CENPEv2, CENPEv3, or CENPEv4 polypeptides, respectively. Identifying the presence of CENPEv2 can be used, for example, to identify cells producing CENPEv2. Such identification provides an additional source of CENPEv2 and can be used to distinguish cells known to produce CENPEv2 from cells that do not produce CENPEv2. For example, antibodies to CENPEv2 can distinguish human cells expressing CENPEv2 from human cells not expressing CENPEv2 or non-human cells (including bacteria) that do not express CENPEv2. Such CENPEv2 antibodies can also be used to determine the effectiveness of CENPEv2 ligands, using techniques well known in the art, to detect and quantify changes in the protein levels of CENPEv2 in cellular extracts, and in situ immunostaining of cells and tissues. In addition, the same above-described utilities also exist for CENPEv3-specific antibodies, and CENPEv4-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CENPEv2, CENPEv3, and CENPEv4 Binding Assay

A number of compounds known to modulate CENPE activity have been disclosed. For example, U.S. Pat. No. 6,489,134 discloses compounds derived from the marine sponge Adocia that are effective modulators of kinesin motors, including CENPE. Adocia derived compounds act by blocking the binding of microtubules to CENPE. Famesyl transferase inhibitors such as SCH 66336 also block the binding of microtubules to CENPE (Ashar, et. al., 2000). Methods for screening compounds for their effects on CENPE activity have also been disclosed. These include microtubule gliding assays, microtubule binding assays, ATPase assays, and microtubule depolymerization assays (Vale, et. al., 1985, Cell 42, 39-50; Kodama, et. al., 1986, J. Biochem. 99, 1465-1472; Stewart, et. al., 1993, Proc. Nat'l. Acad. Sci. 90, 5209-5213; U.S. Pat. No. 6,410,254; Lombillo, et. al., 1995, J. Cell. Biol. 128, 107-115). A person skilled in the art should be able to use these methods to screen CENPEv2, CENPEv3, or CENPEv4 polypeptides for compounds that bind to, and in some cases functionally alter, each respective CENPE isoform protein.

CENPEv2, CENPEv3, or CENPEv4, or fragments thereof, can be used in binding studies to identify compounds binding to or interacting with CENPEv2, CENPEv3, or CENPEv4, or fragments thereof. In one embodiment, the CENPEv2, or a fragment thereof, can be used in binding studies with a CENPE isoform protein, or a fragment thereof, to identify compounds that: bind to or interact with CENPEv2 and other CENPE isoforms; bind to or interact with one or more other CENPE isoforms and not with CENPEv2. A similar series of compound screens can, of course, also be performed using CENPEv3 or CENPEv4 rather than, or in addition to, CENPEv2. Such binding studies can be performed using different formats including competitive and non-competitive formats. Further competition studies can be carried out using additional compounds determined to bind to CENPEv2, CENPEv3, or CENPEv4 or other CENPE isoforms.

The particular CENPEv2, CENPEv3, or CENPEv4 sequence involved in ligand binding can be identified using labeled compounds that bind to the protein and different protein fragments. Different strategies can be employed to select fragments to be tested to narrow down the binding region. Examples of such strategies include testing consecutive fragments about amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using CENPEv2 expressed from a recombinant nucleic acid. Alternatively, recombinantly expressed CENPEv2 consists of the SEQ ID NO 7 amino acid sequence. In addition, binding studies are performed using CENPEv3 expressed from a recombinant nucleic acid. Alternatively, recombinantly expressed CENPEv3 consists of the SEQ ID NO 9 amino acid sequence. In addition, binding studies are performed using CENPEv4 expressed from a recombinant nucleic acid. Alternatively, recombinantly expressed CENPEv4 consists of the SEQ ID NO 11 amino acid sequence.

Binding assays can be performed using individual compounds or preparations containing different numbers of compounds. A preparation containing different numbers of compounds having the ability to bind to CENPEv2, CENPEv3, or CENPEv4 can be divided into smaller groups of compounds that can be tested to identify the compound(s) binding to CENPEv2, CENPEv3, or CENPEv4, respectively.

Binding assays can be performed using recombinantly produced CENPEv2, CENPEv3, or CENPEv4 present in different environments. Such environments include, for example, cell extracts and purified cell extracts containing a CENPEv2, CENPEv3, or CENPEv4 recombinant nucleic acid; and also include, for example, the use of a purified CENPEv2, CENPEv3, or CENPEv4 polypeptide produced by recombinant means which is introduced into different environments.

In one embodiment of the invention, a binding method is provided for screening for a compound able to bind selectively to CENPEv2. The method comprises the steps: providing a CENPEv2 polypeptide comprising SEQ ID NO 7; providing a CENPE isoform polypeptide that is not CENPEv2; contacting the CENPEv2 polypeptide and the CENPE isoform polypeptide that is not CENPEv2 with a test preparation comprising one or more test compounds; and then determining the binding of the test preparation to the CENPEv2 polypeptide and to the CENPE isoform polypeptide that is not CENPEv2, wherein a test preparation that binds to the CENPEv2 polypeptide, but does not bind to CENPE isoform polypeptide that is not CENPEv2, contains one or more compounds that selectively binds to CENPEv2.

In another embodiment of the invention, a binding method is provided for screening for a compound able to bind selectively to CENPEv3. The method comprises the steps: providing a CENPEv3 polypeptide comprising SEQ ID NO 9; providing a CENPE isoform polypeptide that is not CENPEv3; contacting the CENPEv3 polypeptide and the CENPE isoform polypeptide that is not CENPEv3 with a test preparation comprising one or more test compounds; and then determining the binding of the test preparation to the CENPEv3 polypeptide and to the CENPE isoform polypeptide that is not CENPEv3, wherein a test preparation that binds to the CENPEv3 polypeptide, but does not bind to CENPE isoform polypeptide that is not CENPEv3, contains one or more compounds that selectively binds to CENPEv3.

In one embodiment of the invention, a binding method is provided for screening for a compound able to bind selectively to CENPEv4. The method comprises the steps: providing a CENPEv4 polypeptide comprising SEQ ID NO 1; providing a CENPE isoform polypeptide that is not CENPEv4; contacting the CENPEv4 polypeptide and the CENPE isoform polypeptide that is not CENPEv4 with a test preparation comprising one or more test compounds; and then determining the binding of the test preparation to the CENPEv4 polypeptide and to the CENPE isoform polypeptide that is not CENPEv4, wherein a test preparation that binds to the CENPEv4 polypeptide, but does not bind to CENPE isoform polypeptide that is not CENPEv4, contains one or more compounds that selectively binds to CENPEv4.

In another embodiment of the invention, a binding method is provided for screening for a compound able to bind selectively to a CENPE isoform polypeptide that is not CENPEv2. The method comprises the steps: providing a CENPEv2 polypeptide comprising SEQ ID NO 7; providing a CENPE isoform polypeptide that is not CENPEv2; contacting the CENPEv2 polypeptide and the CENPE isoform polypeptide that is not CENPEv2 with a test preparation comprising one or more test compounds; and then determining the binding of the test preparation to the CENPEv2 polypeptide and the CENPE isoform polypeptide that is not CENPEv2, wherein a test preparation that binds the CENPE isoform polypeptide that is not CENPEv2, but does not bind the CENPEv2, contains a compound that selectively binds the CENPE isoform polypeptide that is not CENPEv2. Alternatively, the above method can be used to identify compounds that bind selectively to a CENPE isoform polypeptide that is not CENPEv3 by performing the method with CENPEv3 polypeptide comprising SEQ ID NO 9. Alternatively, the above method can be used to identify compounds that bind selectively to a CENPE isoform polypeptide that is not CENPEv4 by performing the method with CENPEv4 polypeptide comprising SEQ ID NO 11.

The above-described selective binding assays can also be performed with a polypeptide fragment of CENPEv2, CENPEv3, or CENPEv4, wherein the polypeptide fragment comprises at least 10 consecutive amino acids that are coded by a nucleotide sequence that bridges the junction created by the splicing of the 3′ end of CENPEv1 exon 37 to the 5′ end of CENPEv1 exon 39 in the case of CENPEv2, CENPEv3, or CENPEv4; by a nucleotide sequence that bridges the junction created by the splicing of the 3′ end of exon 16 to the 5′ end of exon 18 in the case of CENPEv3; or by a nucleotide sequence that bridges the junction created by the splicing of the 3′ end of exon 16 to the 5′ end of exon 19 in the case of CENPEv4.

Similarly, the selective binding assays may also be performed using a polypeptide fragment of an CENPE isoform polypeptide that is not CENPEv2, CENPEv3, or CENPEv4, wherein the polypeptide fragment comprises at least 10 consecutive amino acids that are coded by: a) a nucleotide sequence that is contained within exon 38 of the CENPEv1 gene; b) a nucleotide sequence that is contained within exon 17 or exon 18 of the CENPE gene; c) a nucleotide sequence that bridges the junction created by the splicing of the 3′ end of exon 37 to the 5′ end of exon 38, or the splicing of the 3′ end of exon 38 to the 5′ end of exon 39 of the CENPEv1 gene; or d) a nucleotide sequence that bridges the junction created by the splicing of the 3′ end of exon 16 to the 5′ end of exon 17, or the splicing of the 3′ end of exon 17 to the 5′ end of exon 18, or the splicing of the 3′ end of exon 18 to the 5′ end of exon 19 of the CENPE gene.

In alternative aspects the above described selective binding assays, compounds maybe screened using the CENPEv2, CENPEv3 or CENPEv4 isoforms using one or more mitotic kinesin protein that are not the respective CENPE isoform instead of a different CENPE isoform. Other mitotic kinesin proteins include, but is not limited to, KSP, KIF4A, KIF14, MPOHOPH1, hklp2, KNSL6, RAB6KIFL, KNSL5, KNSL4, and KNSL1.

CENPE Functional Assays

CENPE is essential to the movement of chromosomes during mitosis. CENPE is a kinetochore associated protein that binds the kinetochore to spindle microtubules. CENPE activity depends on its ability to bind to the kinetochore and microtublules, and on its state of phosphorylation and farnesylation. The identification of CENPEv2, CENPEv3, and CENPEv4 as variants of CENPE provides a means for screening for compounds that bind to CENPEv2, CENPEv3, and/or CENPEv4 protein thereby altering the ability of the CENPEv2, CENPEv3, and/or CENPEv4 polypeptide to bind to the kinetochore complex, to bind to microtubules, or to be phosphorylated or farnesylated. Assays involving a functional CENPEv2, CENPEv3, or CENPEv4 polypeptide can be employed for different purposes, such as selecting for compounds active at CENPEv2, CENPEv3, or CENPEv4; evaluating the ability of a compound to effect the binding of CENPEv2, CENPEv3, or CENPEv4 to the kinetochore or to microtublules, or to effect the phosphorylation or famesylation of CENPEv2, CENPEv3, or CENPEv4; and mapping the activity of different CENPEv2, CENPEv3, and CENPEv4 regions. CENPEv2, CENPEv3, and CENPEv4 activity can be measured using different techniques such as: detecting a change in the intracellular conformation of CENPEv2, CENPEv3, or CENPEv4; detecting a change in the intracellular location of CENPEv2, CENPEv3, or CENPEv4; detecting the amount of binding of CENPEv2, CENPEv3, or CENPEv4 to the kinetochore complex or to microtublues; detecting a change in the alignment of chromosomes or in mitotic progression; or indirectly, by measuring cell apoptosis.

Recombinantly expressed CENPEv2, CENPEv3, and CENPEv4 can be used to facilitate determining whether a compound is active at CENPEv2, CENPEv3, and CENPEv4. For example, CENPEv2, CENPEv3, and CENPEv4 can be expressed by an expression vector in a cell line and used in a co-culture growth assay, such as described in WO 99/59037, to identify compounds that bind to CENPEv2, CENPEv3, and CENPEv4. For example, CENPEv2 can be expressed by an expression vector in a human kidney cell line 293 and used in a co-culture growth assay, such as described in U.S. Patent Application 20020061860, to identify compounds that bind to CENPE v2. A similar strategy can be used for CENPEv3 or CENPEv4.

Techniques for measuring CENPE activity are well known in the art. In addition to the ATPase assays, and microtubule motility, binding, and depolymerization assays described supra, a variety of other assays may be used to investigate the properties of CENPE and therefore would also be applicable to the measurement of CENPEv2, CENPEv3, or CENPEv4 functions. These include immunofluorescence microscopy observation of cells undergoing mitosis (Yen, et. al., 1991), and assays that indirectly measure CENPE activity by measuring cell metabolism and apoptosis, e.g., alamar blue assay (Matute-Bello, et. al., 1999 J. Immunol. 163, 2217-22225); caspase apoptosis assay (BD Biosciences Clontech, Cat. No. K2026-1, Palo alto, Calif.).

CENPEv2, CENPEv3, or CENPEv4 functional assays can be performed using cells expressing CENPEv2, CENPEv3, or CENPEv4 at a high level. These proteins will be contacted with individual compounds or preparations containing different compounds. A preparation containing different compounds where one or more compounds affect CENPEv2, CENPEv3, or CENPEv4 in cells over-producing CENPEv2, CENPEv3, or CENPEv4 as compared to control cells containing expression vector lacking CENPEv2, CENPEv3, or CENPEv4 coding sequences, can be divided into smaller groups of compounds to identify the compound(s) affecting CENPEv2, CENPEv3, or CENPEv4 activity, respectively.

CENPEv2, CENPEv3, or CENPEv4 functional assays can be performed using recombinantly produced CENPEv2, CENPEv3, or CENPEv4 present in different environments. Such environments include, for example, cell extracts and purified cell extracts containing the CENPEv2, CENPEv3, or CENPEv4 expressed from recombinant nucleic acid; and the use of a purified CENPEv2, CENPEv3, or CENPEv4 produced by recombinant means that is introduced into a different environment suitable for measuring kinetochore or microtubule binding; motor activity; mitotic progression; or cell apoptosis.

Modulating CENPEv2, CENPEv3, and CENPEv4 Expression

CENPEv2, CENPEv3, or CENPEv4 expression can be modulated as a means for increasing or decreasing CENPEv2, CENPEv3, or CENPEv4 activity, respectively. Such modulation includes inhibiting the activity of nucleic acids encoding the CENPE isoform target to reduce CENPE isoform protein or polypeptide expressions, or supplying CENPE nucleic acids to increase the level of expression of the CENPE target polypeptide thereby increasing CENPE activity.

Inhibition of CENPEv2, CENPEv3, and CENPEv4 Activity

CENPEv2, CENPEv3, or CENPEv4 nucleic acid activity can be inhibited using nucleic acids recognizing CENPEv2, CENPEv3, or CENPEv4 nucleic acid and affecting the ability of such nucleic acid to be transcribed or translated. Inhibition of CENPEv2, CENPEv3, or CENPEv4 nucleic acid activity can be used, for example, in target validation studies.

A preferred target for inhibiting CENPEv2, CENPEv3, or CENPEv4 is mRNA stability and translation. The ability of CENPEv2, CENPEv3, or CENPEv4 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity. For example, anti-sense oligonucleotides directed to the AUG initiation codon have been shown to almost completely inhibit CENPE and cause long-term mitotic arrest (Yao, et. al. 2000).

RNAi also can be used to prevent protein expression of a target transcript. This method is based on the interfering properties of double-stranded RNA derived from the coding region of a gene that disrupts the synthesis of protein from transcribed RNA. For example, since CENPEv1 exon 38 does not appear to be expressed in normal tissue, but is expressed in at least one human breast cancer cell line, RNAi targeted to sequences within the CENPEv1 exon 38 coding sequence (SEQ ID NO 18) may be a useful therapeutic for breast cancer by inhibiting the synthesis of CENPE proteins that include polypeptides comprising SEQ ID NO 19.

Antibodies directed toward various regions of CENPE, when microinjected into cells can inhibit CENPE activity. For example, mAB 177 (directed to the stalk region), HX-1 (directed to the rod domain) and DraB (directed to the carboxy terminus) all slow or stop mitotic progression (Yen, et. al., 1991; Schaar, et. al., 1997).

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can effect nucleic acid stability. Examples of references describing different anti-sense molecules, and ribozymes, and the use of such molecules, are provided in U.S. Pat. Nos. 5,849,902; 5,859,221; 5,852,188; and 5,616,459.

RNA interference (RNAi) refers to an inhibitory RNA that silences expression of a target protein by RNA interference (McManus & Sharp (2002) Nat. Rev. Genet. 3:737-47; Hannon (2002) Nature 418:244-51; Paddison & Hannon (2002) Cancer Cell 2:17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 21 nucleotides, termed short-interfering RNAs or siRNAs, composed of 19 nucleotides of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

Recently, it was determined that gene silencing could be induced by presenting the cell with the siRNA, mimicking the product of Dicer cleavage (Elbashir et al. (2001) Nature 411:494-8; Elbashir et al. (2001) Genes Dev. 15:188-200). Synthetic siRNA duplexes maintain the ability to associate with RISC and direct silencing of mRNA transcripts, thus providing researchers with a powerful tool for gene silencing in mammalian cells. Yet another method to introduce the dsRNA for gene silencing is shRNA, for short hairpin RNA (Paddison et al. (2002) Genes Dev. 16:948-58; Brummelkamp et al. (2002) Science 296:550-3; Sui et al. (2002) Proc. Natl. Acad. Sci. U.S.A. 99:5515-20). In this case, a desired siRNA sequence is expressed from a plasmid (or virus) as an inverted repeat with an intervening loop sequence to form a hairpin structure. The resulting RNA transcript containing the hairpin is subsequently processed by Dicer to produce siRNAs for silencing. Plasmid-based shRNAs can be expressed stably in cells, allowing long-term gene silencing in cells, or even in animals (McCaffrey et al. (2002) Nature 418:38-9; Xia et al. (2002) Nat. Biotech. 20:1006-10; Lewis et al. (2002) Nat. Genetics 32:107-8; Rubinson et al. (2003) Nat. Genetics 33:401-6; Tiscornia et al. (2003) Proc. Natl. Acad. Sci. U.S.A. 100:1844-8). RNA interference has been successful used therapeutically to protect mice from fulminant hepatitis (Song et al. (2003) Nat. Medicine 9:347-51).

Increasing CENPEv2, CENPEv3, and CENPEv4 Expression

Nucleic acids encoding for CENPEv2, CENPEv3, or CENPEv4 can be used, for example, to cause an increase in CENPE activity or to create a test system (e.g., a transgenic animal) for screening for compounds affecting CENPEv2, CENPEv3, or CENPEv4 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CENPEv2, CENPEv3, and CENPEv4 Using Microarrays

To identify variants in the splicing of the exon regions encoding CENPE, an exon junction microarray, comprising probes complementary to each splice junction resulting from splicing of the 50 exon coding sequences in CENPEv1 heteronuclear RNA (hnRNA), was hybridized to a mixture of labeled nucleic acid samples prepared from 44 different human tissue and cell line samples. Exon junction microarrays are described in PCT patent applications WO 02/18646 and WO 02/16650. Materials and methods for preparing hybridization samples from purified RNA, hybridizing a microarray, detecting hybridization signals, and data analysis are described in van't Veer, et al. (2002 Nature 415:530-536) and Hughes, et al. (2001 Nature Biotechnol. 19:342-7). Inspection of the exon junction microarray hybridization data (not shown) suggested that the structure of at least one of the exon junctions of CENPEv1 mRNA was altered in some of the tissues examined, suggesting the presence of CENPE splice variant mRNA populations. Reverse transcription and polymerase chain reaction (RT-PCR) were then performed using oligonucleotide primer pairs complementary to CENPEv1 exons 13 and 19, and CENPEv1 exons 37 and 39 to confirm the exon junction array results and to allow the sequence structure of the splice variants to be determined.

### Example 2

### Confirmation of CENPEv2 Using RT-PCR

The structure of CENPE mRNA in the region corresponding to CENPEv1 exons 37 to 39 was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 37 and exon 39 of the reference exon coding sequences in CENPEv1 (NM_{—}001813.1). Based upon the nucleotide sequence of CENPEv1 mRNA, the CENPEv1 exon 37 and exon 39 primer set (hereafter CENPE₃₇₋₃₉ primer set) was expected to amplify a 506 base pairs amplicon representing the “reference” CENPEv1 mRNA region. The CENPEv1 exon 37 forward primer has the sequence: 5′ CAACAGGAACTAAAAACTGCTC GTATGC 3′ [SEQ ID NO 20]; and the CENPEv1 exon 39 reverse primer has the sequence: 5′ AGGCTTTCCATAAGGTGCTGTTGTCCAT 3′ [SEQ ID NO 21].

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:
Cycling conditions were as follows:
   50° C. for 30 minutes;
   95° C. for 15 minutes;
   35 cycles of:
      94° C. for 30 seconds;
      63.5° C. for 40 seconds;
      72° C. for 50 seconds; then
      72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons were obtained from human mRNA samples using the CENPE₃₇₋₃₉ primer set (data not shown). Only one of the human tissue and cell lines assayed, testis, had large amounts of the expected amplicon size of 506 base pairs corresponding to the published exon-splicing pattern of CENPEv1 mRNA. Three other samples—leukemia promyelocytic, prostate and epididymus normal—had low amounts of the 506 base pair amplicon. However, all tissue and cell lines assayed, except for interventricular septum normal, which exhibited no PCR product, had large amounts of an amplicon of about 221 base pairs, including those exhibiting the 506 base pair amplicon. The tissues in which CENPEv1 and CENPEv2 mRNAs were detected are listed in Table 1.

TABLE 1

| | | CENPEv2 |
|---|---|---|
| | CENPEv1 | (221 bp |
| Sample | (506 bp amplicon) | amplicon) |
| Heart | | x |
| Kidney | | x |
| Liver | | x |
| Brain | | x |
| Placenta | | x |
| Lung | | x |
| Fetal Brian | | x |
| Leukemia Promyelocytic (HL-60) | x | x |
| Adrenal Gland | | x |
| Fetal Liver | | x |
| Salivary Gland | | x |
| Pancreas | | x |
| Skeletal Muscle | | x |
| Brain Cerebellum | | x |
| Stomach | | x |
| Trachea | | x |
| Thyroid | | x |
| Bone Marrow | | x |
| Brain Amygdala | | x |
| Brain Caudate Nucleus | | x |
| Brain Corpus Callosum | | x |
| Ileocecum | | x |
| Lymphoma Burkitt's (Raji) | | x |
| Spinal Cord | | x |
| Lymph Node | | x |
| Fetal Kidney | | x |
| Uterus | | x |
| Spleen | | x |
| Brain Thalamus | | x |
| Fetal Lung | | x |
| Testis | x | x |
| Melanoma (G361) | | x |
| Lung Carcinoma (A549) | | x |
| Adrenal Medula, normal | | x |
| Brain, Cerebral Cortex, normal; | | x |
| Descending Colon, normal | | x |
| Prostate | x | x |
| Duodenum, normal | | x |
| Epididymus, normal | x | x |
| Brain, Hippocamus, normal | | x |
| Ileum, normal | | x |
| Interventricular Septum, normal | | |
| Jejunum, normal | | x |
| Rectum, normal | | x |

Sequence analysis of the about 221 base pair amplicon, herein referred to as “CENPEv2,” revealed that this amplicon form results from the splicing of exon 37 of the CENPEv1 hnRNA to exon 39; that is, CENPEv1 exon 38 coding sequence is completely absent. Thus, the RT-PCR results confirmed the junction probe microarray data reported in Example 1, which suggested that CENPE mRNA is composed of a mixed population of molecules wherein in at least one of the CENPE mRNA splice junctions is altered.

### Example 3

### Confirmation of CENPEv3 and CENPEv4 Using RT-PCR

The structure of CENPE mRNA in the region corresponding to exons 13 to 19 was determined for a panel of human tissue and cell line samples using an RT-PCR based assay.

PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 13 and exon 19 of the reference exon coding sequences in CENPEv1 (NM_{—}001813.1). Based upon the nucleotide sequence of CENPEv1 mRNA, the CENPEv1 exon 13 and exon 19 primer set (hereafter CENPEv₁₃₋₁₉ primer set) was expected to amplify a 740 base pairs amplicon representing the “reference” CENPEv1 mRNA region. The CENPEv1 exon 13 forward primer has the sequence: 5′ TAACACGGATGCTGGTGACCTCTTCTTC 3′ [SEQ ID NO 22]; and the CENPEv1 exon 19 reverse primer has the sequence: 5′ AAAGGCTG ATTCTCTCTTGGCATCAAGG 3′ [SEQ ID NO 23].

Twenty-five ng of polyA mRNA from each tissue was subjected to a one-step reverse transcription-PCR amplification protocol using the Qiagen, Inc. (Valencia, Calif.), One-Step RT-PCR kit, using the following conditions:
Cycling conditions were as follows:
   50° C. for 30 minutes;
   95° C. for 15 minutes;
   35 cycles of:
      94° C. for 30 seconds;
      63.5° C. for 40 seconds;
      72° C. for 50 seconds; then
      72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons, one of about 665 base pairs, and one of about 545 base pairs, were obtained from human mRNA samples using the CENPE₁₃₋₁₉ primer set (data not shown). The tissues in which CENPEv3 and CENPEv4 mRNAs were detected are listed in Table 2.

TABLE 2

| | | CENPEv4 |
|---|---|---|
| | CENPEv3 | (545 bp |
| Sample | (665 bp amplicon) | amplicon) |
| Heart | | |
| Kidney | x | |
| Liver | x | |
| Brain | x | x |
| Placenta | | |
| Lung | | |
| Fetal Brain | x | |
| Leukemia Promyelocytic (HL-60) | x | |
| Adrenal Gland | | |
| Fetal Liver | x | |
| Salivary Gland | | |
| Pancreas | | |
| Skeletal Muscle | | |
| Brain Cerebellum | x | x |
| Stomach | x | |
| Trachea | x | |
| Thyroid | x | |
| Bone Marrow | x | |
| Brain Amygdala | x | |
| Brain Caudate Nucleus | x | |
| Brain Corpus Callosum | x | |
| Ileocecum | x | |
| Lymphoma Burkitt's (Raji) | x | |
| Spinal Cord | x | |
| Lymph Node | | |
| Fetal Kidney | x | |
| Uterus | | |
| Spleen | | |
| Brain Thalamus | x | |
| Fetal Lung | x | x |
| Testis | x | |
| Melanoma (G361) | x | |
| Lung Carcinoma (A549) | x | |
| Adrenal Medula, normal | | |
| Brain, Cerebral Cortex, normal; | x | x |
| Descending Colon, normal | | |
| Prostate | x | |
| Duodenum, normal | x | |
| Epididymus, normal | | |
| Brain, Hippocamus, normal | | |
| Ileum, normal | x | |
| Interventricular Septum, normal | | |
| Jejunum, normal | | |
| Rectum, normal | x | |

Sequence analysis of the about 665 base pair amplicon, herein referred to as “CENPEv3,” revealed that this amplicon form results from the splicing of exon 16 of the CENPE hnRNA to exon 18; that is, exon 17 coding sequence is completely absent. Sequence analysis of the about 545 base pair amplicon, herein referred to as “CENPEv4,” revealed that this amplicon form results from the splicing of exon 16 of the CENPE hnRNA to exon 19; that is, exon 17 and exon 18 coding sequence is completely absent. Thus, the RT-PCR results confirmed the junction probe microarray data reported in Example 1, which suggested that CENPE mRNA is composed of a mixed population of molecules wherein in at least one of the CENPE mRNA splice junctions is altered.

### Example 4

### Cloning of CENPEv2, CENPEv3, or CENPEv4

Microarray and RT-PCR data indicate that in addition to the CENPEv1 reference mRNA sequence, NM_{—}001813.1, encoding CENPEv1 protein, NP_{—}001804.1, novel splice variant forms of CENPE mRNA, CENPEv2, CENPEv3, and CENPEv4 exists in many tissues, and indeed, CENPEv2 is the form prevalently expressed.

Clones having nucleotide sequence comprising the variants identified in Examples 2 and 3, hereinafter referred to CENPEv2, CENPEv3, or CENPEv4 are isolated using a 5′ “forward” CENPE primer and a 3′ “reverse” CENPE primer, to amplify and clone the entire CENPEv2, CENPEv3, or CENPEv4 mRNA coding sequences, respectively. The 5′ “forward” primer designed for isolation of full length clones corresponding to the CENPEv2, CENPEv3, and CENPEv4 variants has the nucleotide sequence of 5′ ATGGCGGAGGAAGGAGCCGTG GCCGTCT 3′ [SEQ ID NO 24]. The 3′ “reverse” primer designed for isolation of full length clones corresponding to the CENPEv2, CENPEv3, and CENPEv4 variants has the nucleotide sequence of 5′ CTACTGAGTTTTGCACTCAGGCACATCC 3′ [SEQ ID NO 25].

RT-PCR

The CENPEv2, CENPEv3, and CENPEv4 cDNA sequences are cloned using a combination of reverse transcription (RT) and polymerase chain reaction (PCR). More specifically, about 25 ng of fetal brain polyA mRNA (BD Biosciences Clontech, Palo alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the CENPE “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted CENPEv2, CENPEv3, and CENPEv4 mRNAs, products of about 7707, 7632, and 7512 bases, respectively. The remainder of the 50 μl PCR reactions from fetal brain is purified using the QIAquik Gel extraction Kit (Qiagen, Valencia, Calif.) following the QIAquik PCR Purification Protocol provided with the kit. An about 50 μl of product obtained from the purification protocol is concentrated to about 6 μl by drying in a Speed Vac Plus (SC110A, from Savant, Holbrook, N.Y.) attached to a Universal Vacuum Sytem 400 (also from Savant) for about 30 minutes on medium heat.

Cloning of RT-PCR Products

About 4 μl of the 6 μl of purified CENPEv2, CENPEv3, and CENPEv4 RT-PCR products from fetal brain are used in a cloning reaction using the reagents and instructions provided with the TOPO TA cloning kit (Invitrogen, Carlsbad, Calif.). About 2 μl of the cloning reaction is used following the manufacturer's instructions to transform TOP 10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2× LB medium. These liquid cultures are incubated overnight on a roller at 37° C. Plasmid DNA is extracted from these cultures using the Qiagen (Valencia, Calif.) Qiaquik Spin Miniprep kit. Twelve putative CENPEv2, CENPEv3, and CENPEv4 clones, respectively, are identified and prepared for a PCR reaction to confirm the presence of the expected CENPEv2 exon 37 to exon 39, CENPEv3 exon 16 to exon 18, and CENPEv4 exon 16 to exon 19 variant structures. A 25 μl PCR reaction is performed as described above (RT-PCR section) to detect the presence of CENPEv2, except that the reaction includes miniprep DNA from the TOPO TA/CENPEv2 ligation as a template. An additional 25 μl PCR reaction is performed as described above (RT-PCR section) to detect the presence of CENPEv3, except that the reaction includes miniprep DNA from the TOPO TA/CENPEv3 ligation as a template. An additional 25 μl PCR reaction is performed as described above (RT-PCR section) to detect the presence of CENPEv4, except that the reaction includes miniprep DNA from the TOPO TA/CENPEv4 ligation as a template. About 10 μl of each 25 μl PCR reaction is run on a 1% Agarose gel and the DNA bands generated by the PCR reaction are visualized and photographed on a UV light box to determine which minipreps samples have PCR product of the size predicted for the corresponding CENPEv2, CENPEv3, and CENPEv4 variant mRNAs. Clones having the CENPEv2 structure are identified based upon amplification of an amplicon band of 7707 basepairs, whereas a reference CENPEv1 clone will give rise to an amplicon band of 7992 basepairs. Clones having the CENPEv3 structure are identified based upon amplification of an amplicon band of 7632. Clones having the CENPEv4 structure are identified based upon amplification of an amplicon band of 7512 basepairs. DNA sequence analysis of the CENPEv2, CENPEv3, or CENPEv4 cloned DNAs confirm a polynucleotide sequence representing the deletion of exon 38 of the CENPEv1 reference transcript in the case of CENPEv2, CENPEv3, and CENPEv4; the deletion of exon 17 in the case of CENPEv3; and the deletion of exon 17 and exon 18 in the case of CENPEv4.

The polynucleotide sequence of CENPEv2 mRNA (SEQ ID NO 6) contains an open reading frame that encodes a CENPEv2 protein (SEQ ID NO 7) similar to the reference CENPEv1 protein (NP_{—}001804.1), but lacking the amino acids encoded by a 285 base pair region corresponding to exon 38 of the full length coding sequence of reference CENPEv1 mRNA (NM_{—}001813.1). The deletion of the 285 base pair region results in a protein translation reading frame that is in alignment in comparison to the reference CENPEv1 protein reading frame. Therefore, the CENPEv2 protein is only missing an internal 95 amino acid region as compared to the reference CENPEv1 protein (NP_{—}001804.1).

The polynucleotide sequence of CENPEv3 mRNA (SEQ ID NO 8) contains an open reading frame that encodes a CENPEv3 protein (SEQ ID NO 9) similar to the reference CENPEv1 protein (NP_{—}001804.1), but lacking the amino acids encoded by a 285 base pair region corresponding to exon 38, and a 75 base pair region corresponding to exon 17 of the full length coding sequence of reference CENPEv1 mRNA (NM_{—}001813.1). The deletion of the 285 base pair region and the 75 base pair region results in a protein translation reading frame that is in alignment in comparison to the reference CENPEv1 protein reading frame. Therefore the CENPEv3 protein is only missing an internal 95 amino acid region and an internal 25 amino acid region as compared to the reference CENPEv1 protein (NP_{—}001804.1).

The polynucleotide sequence of CENPEv4 mRNA (SEQ ID NO 10) contains an open reading frame that encodes a CENPEv4 protein (SEQ ID NO 11) similar to the reference CENPEv1 protein (NP_{—}001804.1), but lacking the amino acids encoded by a 285 base pair region corresponding to exon 38, and a 195 base pair region corresponding to exon 17 and exon 18 of the full length coding sequence of reference CENPEv1 mRNA (NM_{—}001813.1). The deletion of the 285 base pair region and a 195 base pair region results in a protein translation reading frame that is in alignment in comparison to the reference CENPEv1 protein reading frame. Therefore the CENPEv4 protein is only missing an internal 95 amino acid region and an internal 65 amino acid region as compared to the reference CENPEv1 protein (NP_{—}001804.1).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 6, or the complement thereof.

2. The purified nucleic acid of claim 1, wherein said nucleic acid comprises a region encoding SEQ ID NO 7.

3. The purified nucleic acid of claim 1, wherein said nucleotide sequence encodes a polypeptide consisting of SEQ ID NO 7.

4. A purified polypeptide comprising SEQ ID NO 7.

5. The polypeptide of claim 4, wherein said polypeptide consists of SEQ ID NO 7.

6. An expression vector comprising a nucleotide sequence encoding SEQ ID NO 7, wherein said nucleotide sequence is transcriptionally coupled to an exogenous promoter.

7. The expression vector of claim 6, wherein said nucleotide sequence encodes a polypeptide consisting of SEQ ID NO 7.

8. The expression vector of claim 6, wherein said nucleotide sequence comprises SEQ ID NO 6.

9. The expression vector of claim 6, wherein said nucleotide sequence consists of SEQ ID NO 6.

10. A method for screening for a compound able to bind to CENPEv2, comprising the steps of:
(a) expressing a polypeptide comprising SEQ ID NO 7 from recombinant nucleic acid;
(b) providing to said polypeptide a test preparation comprising one or more test compounds; and
(c) measuring the ability of said test preparation to bind to said polypeptide.

11. The method of claim 10, wherein said steps (b) and (c) are performed in vitro.

12. The method of claim 10, wherein said steps (a), (b), and (c) are performed using a whole cell.

13. The method of claim 10, wherein said polypeptide is expressed from an expression vector.

14. The method of claim 10, wherein said polypeptide consists of SEQ ID NO 7.

15. A method of screening for compounds able to bind selectively to CENPEv2 comprising the steps of:
(a) providing a CENPEv2 polypeptide comprising SEQ ID NO 7;
(b) providing one or more CENPE isoform polypeptides that are not CENPEv2;
(c) contacting said CENPEv2 polypeptide and said CENPE isoform polypeptide that is not CENPEv2 with a test preparation comprising one or more compounds; and
(d) determining the binding of said test preparation to said CENPEv2 polypeptide and to said CENPE isoform polypeptide that is not CENPEv2, wherein a test preparation that binds to said CENPEv2 polypeptide, but does not bind to said CENPE polypeptide that is not CENPEv2, contains a compound that selectively binds said CENPEv2 polypeptide.

16. The method of claim 15, wherein said CENPEv2 polypeptide is obtained by expression of said polypeptide from an expression vector comprising a polynucleotide encoding SEQ ID NO 7.

17. The method of claim 16, wherein said polypeptide consists of SEQ ID NO 7.

18. A method for screening for a compound able to bind to or interact with a CENPEv2 protein or a fragment thereof comprising the steps of:
(a) expressing a CENPEv2 polypeptide comprising SEQ ID NO 7 or fragment thereof from a recombinant nucleic acid;
(b) providing to said polypeptide a labeled CENPE ligand that binds to said polypeptide and a test preparation comprising one or more compounds; and
(c) measuring the effect of said test preparation on binding of said labeled CENPE ligand to said polypeptide, wherein a test preparation that alters the binding of said labeled CENPE ligand to said polypeptide contains a compound that binds to or interacts with said polypeptide.

19. The method of claim 18, wherein said steps (b) and (c) are performed in vitro.

20. The method of claim 18, wherein said steps (a), (b) and (c) are performed using a whole cell.

21. The method of claim 18, wherein said polypeptide is expressed from an expression vector.

22. The method of claim 18, wherein said CENPEv2 ligand is a CENPE inhibitor.

23. The method of claim 21, wherein said expression vector comprises SEQ ID NO 6 or a fragment of SEQ ID NO 6.

24. The method of claim 21, wherein said polypeptide comprises SEQ ID NO 7 or a fragment of SEQ ID NO 7.
